(19) United States
(12) Patent Application Publication
(10) Pub. No.: US 2015/0112716 A1
(43) Pub. Date: Apr. 23, 2015

(54) INDIVIDUALIZED HEALTHCARE MANAGEMENT SYSTEM

(71) Applicants: Enid M. Hunkeler, Oakland, CA (US); Joseph F. Terdiman, San Rafael, CA (US)

(72) Inventors: Enid M. Hunkeler, Oakland, CA (US); Joseph F. Terdiman, San Rafael, CA (US)

(21) Appl. No.: 14/589,342

(22) Filed: Jan. 5, 2015

Related U.S. Application Data
(63) Continuation of application No. 11/155,967, filed on Jun. 17, 2005.
(60) Provisional application No. 60/581,755, filed on Jun. 21, 2004.

(51) Int. Cl. G06F 19/00

(52) U.S. Cl. CPC G06F 19/345; G06F 19/363
USPC 705/2

(57) ABSTRACT

Method and system for providing individualized healthcare to a patient having a chronic disease over the Internet are disclosed. The system includes at least a computer server for executing application programs where the computer server includes storage means for storing patient information in at least a database server and means for communicating with at least a patient computer and a medical provider computer. The system further includes means for providing individualized web pages for interacting with the patient, means for monitoring signs and symptoms of the chronic disease using inputs provided by the patient, and means for administrating an individualized treatment of the chronic disease according to the signs and symptoms provided by the patient.

### CROSS-REFERENCE TO RELATED APPLICATION

This application claims the benefit of U.S. provisional application No. 60/581,755, “Individualized Healthcare Management System” filed Jun. 21, 2004; which is incorporated herein in its entirety by reference. This application is related to the patent application entitled “System and Method for Assisting a Care Partner in Monitoring a Patient with Chronic Disease”, attorney docket number 59559-2000200, which is filed on the same date as this application and is hereby incorporated by reference in its entirety.

### FIELD OF THE INVENTION

The present invention relates to the field of healthcare management. In particular, the present invention relates to an individualized healthcare management system.

### BACKGROUND OF THE INVENTION

Telemedicine refers to the practice of medicine in which the patient and provider are in different locations and communicate electronically. Today, telemedicine usually refers to the use of a telecommunication and/or computer system to transfer information between patient and provider in different locations. With the advent of the Internet and the World Wide Web, such systems commonly take the form of Web-based applications that permit clinical information to be communicated between distant locations. There are two basic modes of telemedicine communication, real-time and store-and-forward. Real-time communication between patient and provider can take the form of audio, video, or instant text messaging. Store-and-forward communication is a form of asynchronous communication between patient and provider. An example of store-and-forward communication is electronic mail.

While most visionaries would agree that telemedicine will play a major role in the delivery of health care in the 21st century, the adoption of telemedicine continues to be a slow process. One of the reasons is that most telemedicine models have been aimed to deliver traditional healthcare, but at a distance. The primary focus of the existing telemedicine models is to cut cost. Such telemedicine models lack individual patient participation and do not foster patients' continued reliance on receiving treatment and managing their chronic diseases. Therefore, there is a need for a healthcare management system that delivers individualized healthcare to patients.

### SUMMARY

Disclosed is an individualized healthcare management system that integrates monitoring of the patient's clinical status with tailored education, individualized action plans, and behavioral modification. The system integrates various healthcare functional components into a single telemedicine package in order to: 1) monitor signs and symptoms of the disease; 2) monitor medication compliance and adverse reactions; 3) display relevant clinical data from legacy systems; 4) provide interactive, individualized patient education on the disease process and treatment; 5) enable care partner support and participation; 6) generate automatic alerts to providers when certain monitoring or compliance criteria indicate that a patient needs help; and 7) provide multiple communication methods between provider and patient that not only give clinicians opportunities to intervene before a health problem becomes a health crisis; they also give patients opportunities to work out self-management strategies to effectively deal with triggers, early warning signs, and symptoms. In addition, the system includes the ability to customize the web site content to the disease or condition, as well as to the needs of the individual patient. The system is adaptive to the patient's increased knowledge of the disease, his own health condition, and his behavior. As the patient becomes aware of a new symptom, early warning sign, helpful response, or harmful response and enters such information into the system, his personal profiles, action plans, and vital signs for regular monitoring are automatically updated to reflect the new knowledge the patient has acquired. By combining the above features into a single telemedicine system accessible from home, patients may benefit from the value of the individualized management and education about their condition. Similarly, providers may benefit from the system both as a resource for providing education to their patients, and as a clinical tool for monitoring the patients' health status and delivering treatments to the patients.

In one embodiment, a method for providing individualized healthcare over the Internet to a patient having a chronic disease includes providing individualized web pages for interacting with the patient, monitoring signs and symptoms of the chronic disease using inputs provided by the patient, and administrating an individualized treatment of the chronic disease according to the signs and symptoms provided by the patient.

In another embodiment, a system for providing individualized healthcare over the Internet to a patient having a chronic disease includes at least a computer server for executing application programs where the computer server includes means for storing patient information in at least a database server and means for communicating with at least a patient computer and a medical provider computer. The system further includes means for providing individualized web pages for interacting with the patient, means for monitoring signs and symptoms of the chronic disease using inputs provided by the patient, and means for administrating an individualized treatment of the chronic disease according to the signs and symptoms provided by the patient.

### BRIEF DESCRIPTION OF THE DRAWINGS

The aforementioned features and advantages of the invention as well as additional features and advantages thereof will be more clearly understood hereinafter as a result of a detailed description of embodiments of the invention when taken in conjunction with the following drawings.

FIG. 1 illustrates an implementation of the eCare system according to an embodiment of the present invention.

FIG. 2 illustrates a patient homepage for accessing individualized healthcare according to an embodiment of the present invention.

FIG. 3 illustrates a nurse homepage for monitoring patients' healthcare according to an embodiment of the present invention.

FIG. 4 illustrates a webpage for inputting patient's symptoms according to an embodiment of the present invention.

FIG. 5 illustrates a webpage for monitoring patient's bipolar symptoms according to an embodiment of the present invention.

FIG. 6 illustrates a webpage for monitoring patient's helpful responses according to an embodiment of the present invention.

FIG. 7 illustrates a webpage for monitoring patient's use of medications according to an embodiment of the present invention.

FIG. 8 illustrates a webpage for providing online education to patients according to an embodiment of the present invention.

FIG. 9 illustrates a webpage for monitoring patient's vital signs according to an embodiment of the present invention.

FIG. 10 is a webpage showing a patient's Action Plan according to an embodiment of the present invention.

FIG. 11 illustrates a webpage for helping a patient in need of assistance according to an embodiment of the present invention.

FIG. 12 illustrates a webpage for helping a patient who cannot sleep according to an embodiment of the present invention.

FIG. 13 illustrates a webpage for sending secure messages between a patient and a healthcare provider according to an embodiment of the present invention.

FIG. 14 illustrates a webpage for managing patient's personal database according to an embodiment of the present invention.

FIG. 15 illustrates an implementation of a care partner home page according to an embodiment of the present invention.

FIG. 16 illustrates a profile of the patient that may be provided by a care partner showing depression symptoms and early warning signs according to an embodiment of the present invention.

FIG. 17 illustrates a profile of the patient that may be provided by a care partner showing social and behavioral functions and warning signs according to an embodiment of the present invention.

FIG. 18 illustrates a web page for monitoring social and behavioral signs of the patient according to an embodiment of the present invention.

FIG. 19 illustrates a web page for monitoring medications and their related side effects on the patient according to an embodiment of the present invention.

FIG. 20 illustrates a webpage for displaying patient's health summary according to an embodiment of the present invention.

FIG. 21 illustrates a webpage for displaying a summary of patient's self monitoring inputs over a period of time according to an embodiment of the present invention.

FIG. 22 illustrates a webpage for monitoring patients' usage of the eCare system according to an embodiment of the present invention.

FIG. 23 illustrates a webpage for monitoring care partners' usage of the eCare system according to an embodiment of the present invention.

FIG. 24 illustrates a webpage for monitoring patients' usage of the eCare system on a particular day of the week according to an embodiment of the present invention.

FIG. 25 illustrates a webpage for monitoring patients' usage of the eCare system on a particular hour of the day according to an embodiment of the present invention.

### DESCRIPTION OF EMBODIMENTS

Methods and systems are provided for an individualized healthcare management system. The following descriptions are presented to enable any person skilled in the art to make and use the invention. Descriptions of specific embodiments and applications are provided only as examples. Various modifications and combinations of the examples described herein will be readily apparent to those skilled in the art, and the general principles defined herein may be applied to other examples and applications without departing from the spirit and scope of the invention. Thus, the present invention is not intended to be limited to the examples described and shown, but is to be accorded the widest scope consistent with the principles and features disclosed herein.

### Treating Bipolar Patients

In one embodiment, a generalized, interactive web-based patient care system for providing individualized healthcare management of bipolar patients is disclosed. This system is also referred to as the eCare system. The eCare system can be generalized to manage and monitor a wide variety of chronic diseases and health conditions in order to: 1) give each patient the right treatment at the right dose for the right duration; 2) maximize each patient's self-management skills; and 3) maximize each patient's ability to create a supportive environment. The eCare system combines monitoring of the patient's clinical status with tailored education, individualized action plans, behavioral modification, and care partner (e.g., family member) support. The eCare system integrates seven major functional components into a single telemedicine package in order to: 1) monitor signs and symptoms of the disease; 2) monitor medication compliance and adverse reactions; 3) display relevant clinical data from legacy systems; 4) provide interactive, individualized patient education on the disease process and treatment; 5) enable care partner support and participation; 6) generate automatic alerts to providers when monitoring or treatment compliance criteria are not met; and 7) provide multiple communication modalities between provider and patient that not only give clinicians opportunities to intervene before a health problem becomes a health crisis; they also give patients opportunities to work out self-management strategies to effectively deal with triggers, early warning signs, and symptoms. The eCare system also includes a calendar for scheduled activities, a task list, a treatment plan, and a health status summary. In addition, the eCare system includes the ability to customize the web site content to a given type of health care delivery, as well as to the needs of the individual patient. The system is adaptive to the patient's increased knowledge of the disease, his own health condition, and his behavior. As the patient becomes aware of a new symptom, early warning sign, helpful response, or harmful response and enters such information into the system, his personal profiles, action plans, and vital signs for regular monitoring are automatically updated to reflect the new knowledge the patient has acquired. On the provider side, the program organizes the workload of the providers, directing them to patients with the greatest need and giving them access to the relevant patient data from the eCare and legacy databases. By combining these features into a single telemedicine system, the eCare system allows patients to access most of the health care services they need in the comfort of their homes and at any time they choose. Patients may appreciate the value of telemedicine. Similarly, providers may use it as both an educational resource for patients and as a clinical tool for monitoring patients' health status and prioritizing their needs.

According to one implementation of the eCare system of the present invention, patients who meet certain disease criteria are managed by nurses, who follow evidence-based protocols, and are supervised by physicians who are experts in those conditions. The treatment protocols are developed by a team of health care providers responsible for the clinic, and generally follow national consensus guidelines for treatment of disease. Nurses, who are trained in the protocols, check their panel of patients periodically by telephone or through a patient visit to assess their clinical status. Treatment is coordinated and given by a psychiatrist as needed. Changes in medication or other treatments usually follow the protocol. It may be effective to manage chronic diseases through this specialized website. In addition, personalization of and improving access to healthcare increase patient satisfaction with the eCare program, this in turn may improve patients' compliance with treatment regimens.

In one approach, the eCare system personalizes and customizes the disease management program for the individual patient, based on the patient's health status, knowledge, and ability to self-manage the disease and treatment. It is imperative that the patient receives positive reinforcement whenever a monitoring task or component of the treatment regimen is completed. The eCare system is integrated into the continuum of care for disease management, without inundating providers with an overwhelming amount of patient information, while, at the same time, sorting the information by urgency of attention required. The eCare system may also be used proactively in helping patients to understand and deal with triggers, early warning signs, and symptoms. For example, if a patient fails to access the web site on schedule, reports side effects from medication, or fails to refill a prescription at the time the dose frequency and quantity dispensed predict the patient may run out of medication, an alert is sent to the provider. A patient may access the web for routine monitoring and education, for specific problems as they arise, or for potentially emergent situations. Triage logic within the application routes the information to the database, sends a routine or urgent message to the provider, or displays instructions on how to reach emergency services by telephone.

The following describes an implementation of the eCare system in providing individualized healthcare management in treating patients with chronic diseases.

### System Configuration

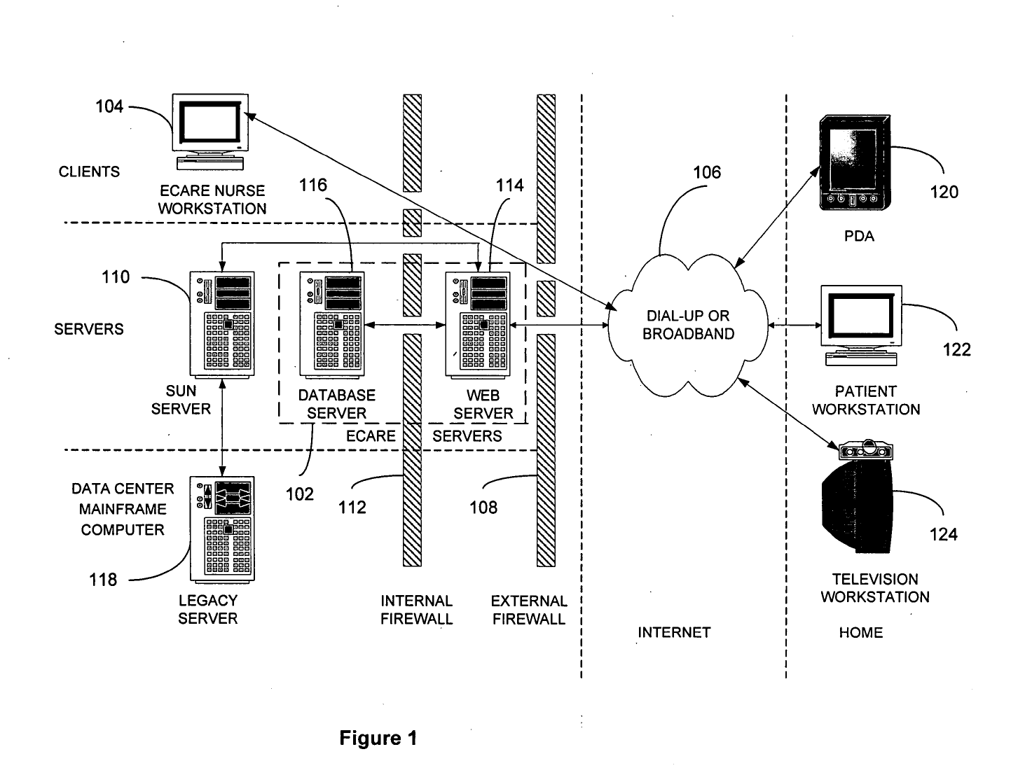

FIG. 1 illustrates an implementation of the eCare system according to an embodiment of the present invention. The eCare system is installed on a local area network, with one or more servers 102 and one or more client workstations 104. The network is connected to the Internet 106 via a broadband connection through an external firewall 108. The one or more servers are PCs and Sun servers, which use Windows, Solaris, and Linux operating systems. A Sun Server 110 is used for data management and statistical analysis. The Sun Server may also communicate with other data centers or main frame computers which support a Legacy Server 118 for storing patients' medical records. Production and development Web Servers 114 are supported between an internal firewall 112 and an external firewall 108. Web Servers 114 used are Apache or IIS web systems. Database Servers 116 supported are Oracle, Microsoft Structured Query Language (SQL), and MySQL database systems.

The eCare system is configured to treat specific chronic diseases and deliver health information on specific topics, with minimum modifications to the software. The configuration is primarily table driven, and supports importing of menus, tables, business logic, database schema, and contents of web pages. Configurable menus and tables include lists of signs and symptoms, physiological monitoring parameters, list of medications, educational programs, treatment protocols, links to other web sites, alert generation criteria, and legacy system interfaces.

The eCare system is developed as a three-tiered open-architecture web application, with a front-end Web Server 114 containing the telemedicine application and business logic, and a back-end Database Server 116 containing an SQL-compliant database management system. The eCare system is designed to be able to operate on the networks of most health care institutions. Many of these institutions have “locked-down” desktop images, whose policies often include the removal of such basic Windows functions as the Run command, the ability to map additional drives, the ability to download new versions of installed applications and Active-X controls (required by many modern Internet Explorer applications). They also include an internal firewall 112 and an external firewall 108 that block a variety of Transmission Control Protocol (TCP) and User Datagram Protocol (UDP) services, required by such applications as voice or video over Internet Protocol (IP). Many institutions also specify a limited set of standard software products that are acceptable for use on the system. To use a non-standard product, these institutions often require a formal waiver by its Information Technology department, which can lead to interminable delays in its deployment. Recognizing these limitations and constraints, the eCare system is designed to use the platforms and software that are supported by most medical institutions.

The database, which contains patients' clinical data and various tables used by the application, resides on an SQL-compliant database server (e.g., Microsoft SQL or Oracle), but may be designed to use any generic SQL database management system. The Database Server 116 may be dedicated to the telemedicine application, or shared by multiple applications. The Database Server is located inside the internal firewall 112 and password protected to prevent unauthorized access. In addition, the database is backed up to disk on another platform and/or tape on a regular basis (e.g., hourly or daily).

The web application runs on a dedicated Web Server 114, which is located in the institution, but outside the internal firewall 112 on an “untrusted” subnet, so that it can be accessed through the Internet 106. The Web Server 114 uses a Secure Sockets Layer (SSL) 128-bit encryption to display web pages on workstations both inside and outside the external firewall 108. The Web Server 114 communicates with the Database Server 116 using IP address-and-port-specific tunnels through the internal firewall 112. Patient data may be cached temporarily on the web browser and deleted at the end of the session. The eCare application is designed to operate over a wide range of user platforms, operating systems, and web browser versions. This is especially important to maximize the number of patients who can run the eCare application successfully, and whose home computers are likely to have the greatest variability in age, speed, capacity, and browser version. The application may be written in a scripting language with the widest portability and compatibility with various computer platforms (e.g., Java). In case of failure of either Web or Database Server, backup servers for both servers may be configured for rapid deployment and restoration of the application.

The eCare application is also designed to work with the client's display screen form factor, in order to be able to operate on wireless and handheld devices, such as tablets and PDAs. As wireless networks expand across the country, an increasing number of eCare transactions by both provider and patient may originate from wireless access points. As shown in FIG. 1, the eCare system supports devices of various formats, such as PDA 120, workstation/personal computer 122, or television workstation 124.

### Patient Homepage and Nurse Homepage

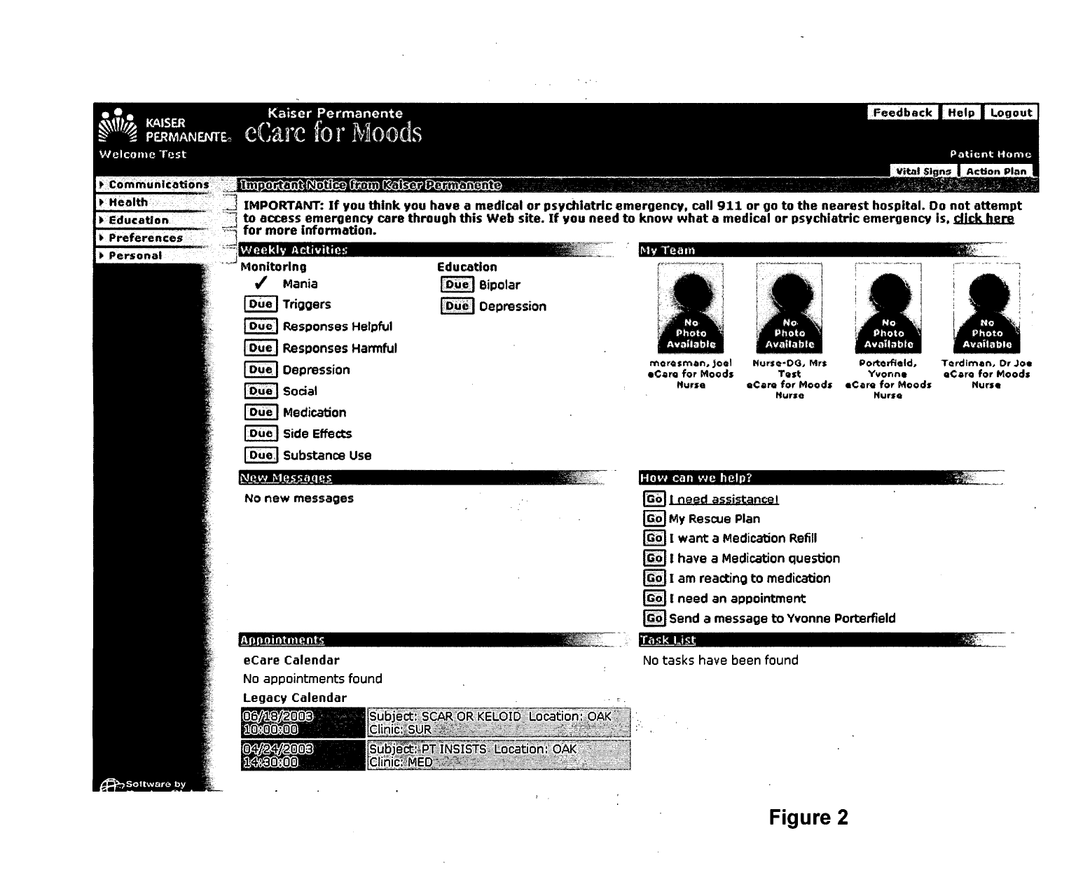

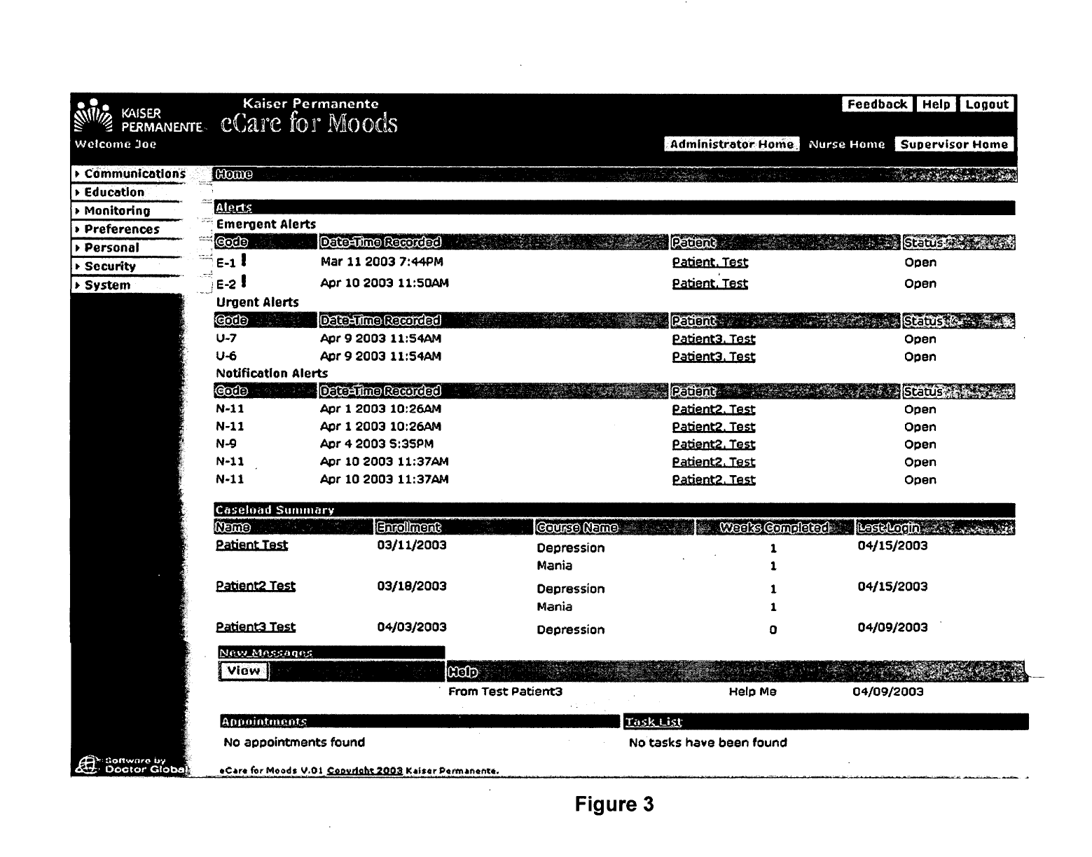

FIG. 2 illustrates a patient homepage for accessing individualized healthcare according to an embodiment of the present invention. A patient's homepage is the place that the patient comes to after having logged into the system. There are various types of information the patient may access from the homepage, including Weekly Activities, My Team, New Messages, How Can We Help, Appointments, and Task List. FIG. 3 illustrates a nurse homepage for monitoring patients' healthcare according to an embodiment of the present invention. The nurse homepage includes Alerts, Caseload Summary, New Messages, Appointments, and Task List. The Alerts further includes Emergent Alerts, Urgent Alerts, and Notification Alerts. Each type of Alert requires different priority of attention from the nurse.

### Monitoring of Symptoms

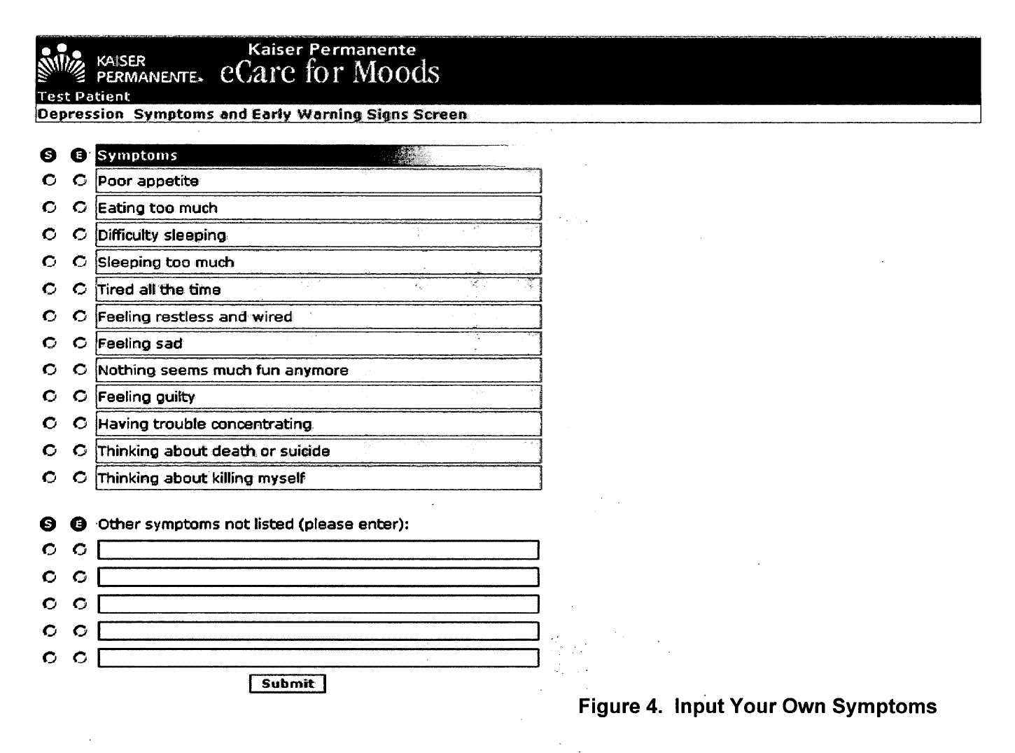

FIG. 4 illustrates a webpage for inputting patient's symptoms according to an embodiment of the present invention. Patients are asked to complete questionnaires about their health or the signs and symptoms of their conditions on a regular basis (e.g., weekly or monthly). A predefined list of symptoms according to medical standards and guidelines is created by the medical team responsible for the program. The list is customized for each patient by the provider, in consultation with the patient and his care partner if one is available. For each symptom listed, the patient selects whether it is an existing symptom or it is an early warning sign or neither. The patient may add other symptoms that are not listed in the predefined list of symptoms to personalize his profile. Additional symptoms that have particular relevance to the patient may be added by provider or patient. Note that this monitoring system is adaptive to the patient's individualized situation. For example, as the patient learns a new symptom, early warning sign, helpful response, or harmful response and enters such information into the system, his personal profiles, action plans, and vital signs for regular monitoring are automatically updated to reflect the new knowledge the patient has acquired.

### Physiological Monitoring

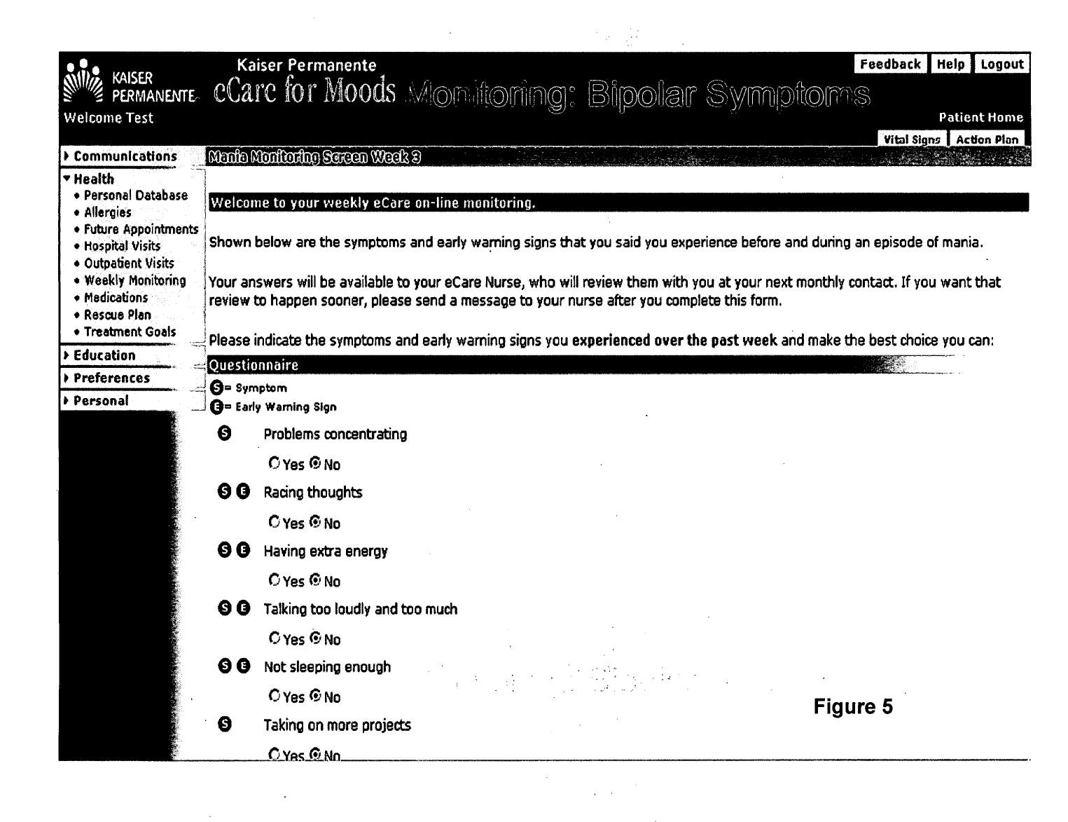

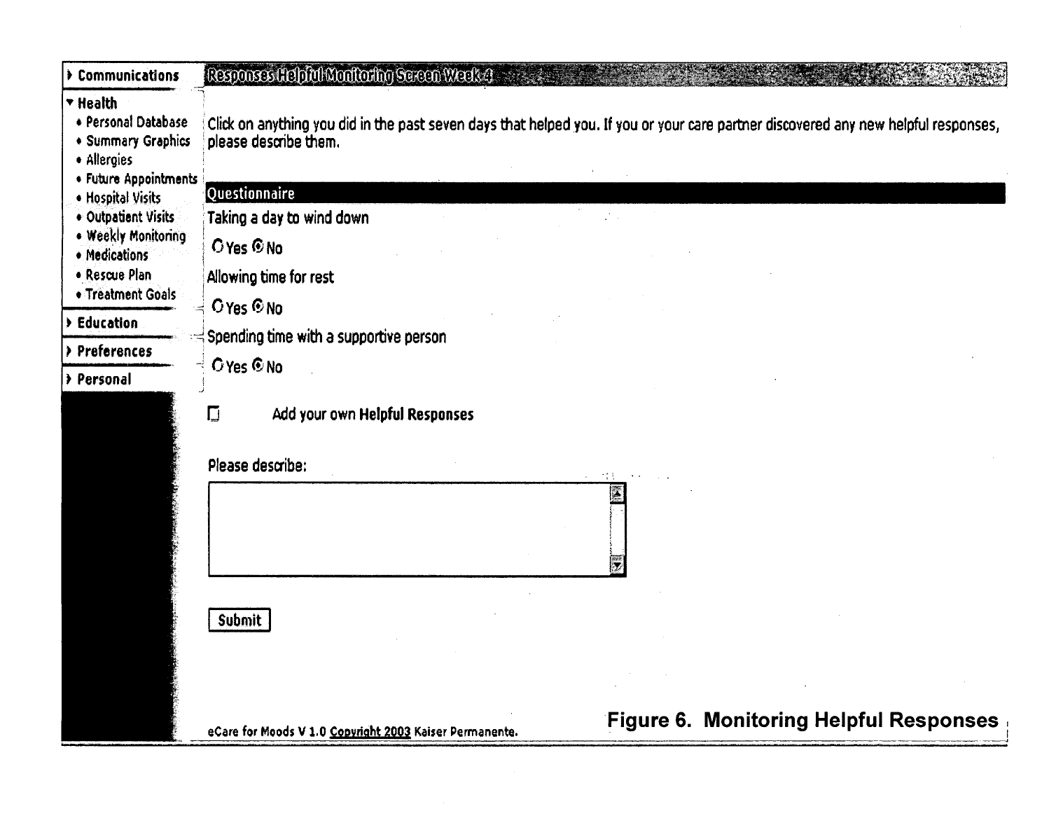

Patients are asked to monitor physiological parameters that are relevant to their particular conditions (e.g., weight, blood sugar, etc.). Patients may use home measuring instruments (e.g., scale, glucometer, sphygmomanometer, etc.), for taking measurements and the results are entered into their home computers while accessing the telemedicine web site. Instruments with a digital interface that enables the measurements to be recorded automatically by the computer program are preferred. Alternatively, the patient may enter the measurements using a keyboard or other devices. A graphical display of measurements over time is developed and may be displayed by patient and provider for monitoring purposes. FIG. 5 illustrates a webpage for monitoring patient's bipolar symptoms according to an embodiment of the present invention; and FIG. 6 illustrates a webpage for monitoring patient's helpful responses according to an embodiment of the present invention. These web pages are examples of self-management tools that the patient may use to effectively monitor triggers, early warning signs, symptoms, helpful and harmful responses.

### Medication Monitoring and Compliance

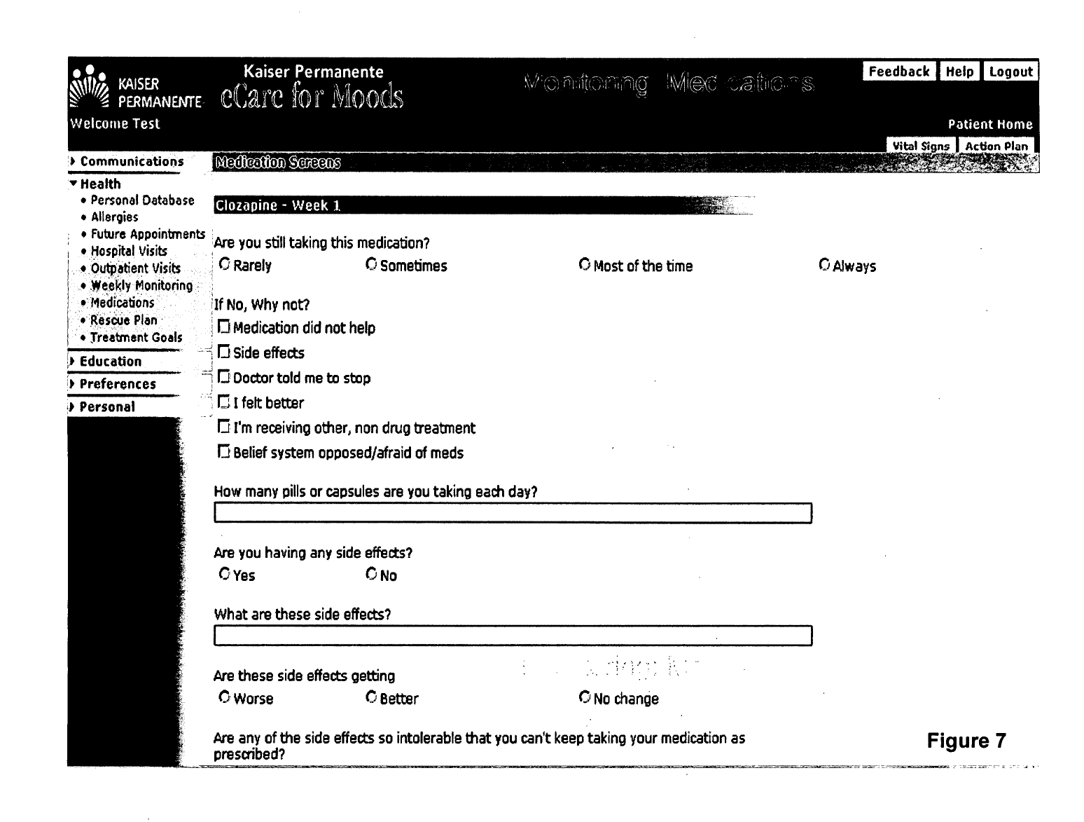

FIG. 7 illustrates a webpage for monitoring patient's use of medications according to an embodiment of the present invention. The eCare system monitors medications taken, including prescribed and over-the-counter medications or herbal supplements. Prescription data, including dosage, frequency, quantity dispensed, and refill status, may be downloaded from legacy systems or entered directly into the eCare system by the provider. When a prescribed medication is due to be refilled, a reminder is automatically sent to the patient to request a refill. Failure by the patient to confirm that the medication has been refilled may result in an alert to the provider. Side effects of medications may be monitored through questionnaires on a regular basis. Certain answers such as ones indicating that the patient is experiencing side effects may also result in an alert to the provider.

### Educational Programs

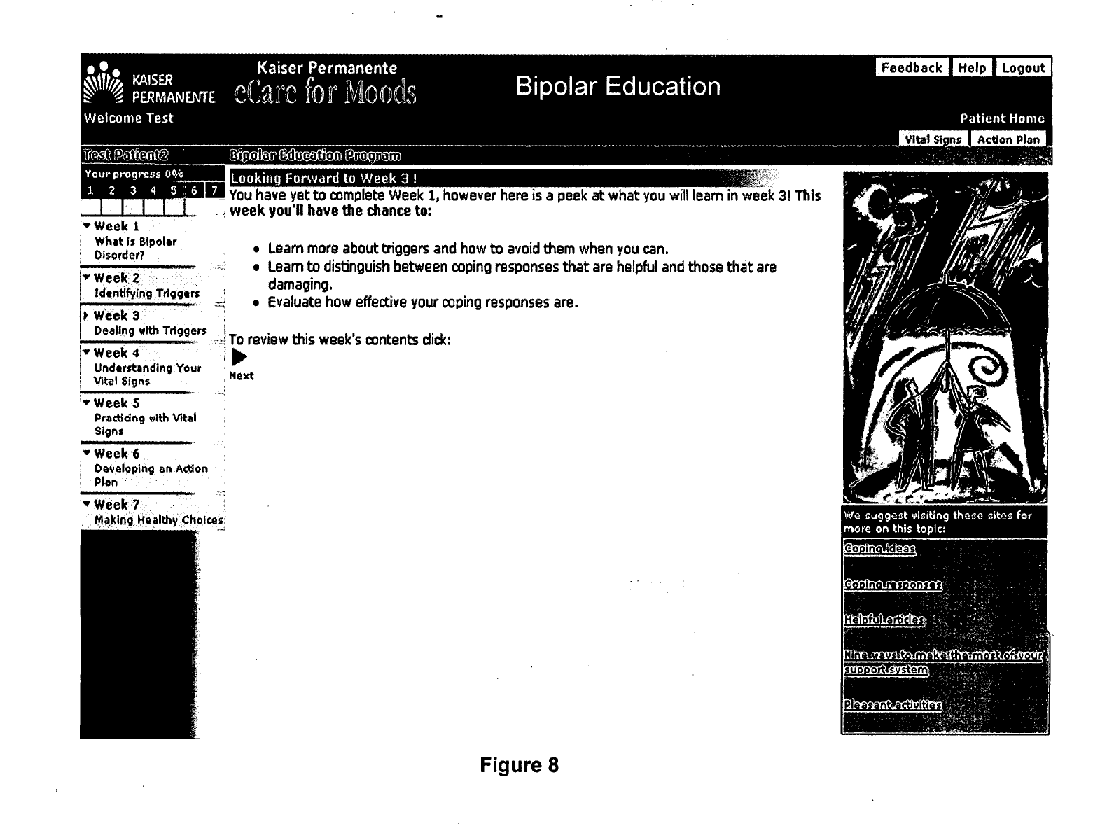

FIG. 8 illustrates a webpage for providing online education to patients according to an embodiment of the present invention. The eCare system includes online, interactive patient education courses that are developed to give patients insight into their condition and treatment. Information presented to the patient in the educational program may also be used to update and personalize the lists of questions in the monitoring program, the patient's personal databases, list of vital signs, and action plans. These interactive individualized health education programs tailored to the specific needs of the patient are the best way to optimize patients' health. Links to additional educational topics, such as coping ideas, coping responses, helpful articles, ways to make the most of your support system, and pleasant activities may also be found on the eCare website. A point system may be established where both the patient and the care partner can be rewarded for completion of certain sections of the program.

### Monitoring Vital Signs

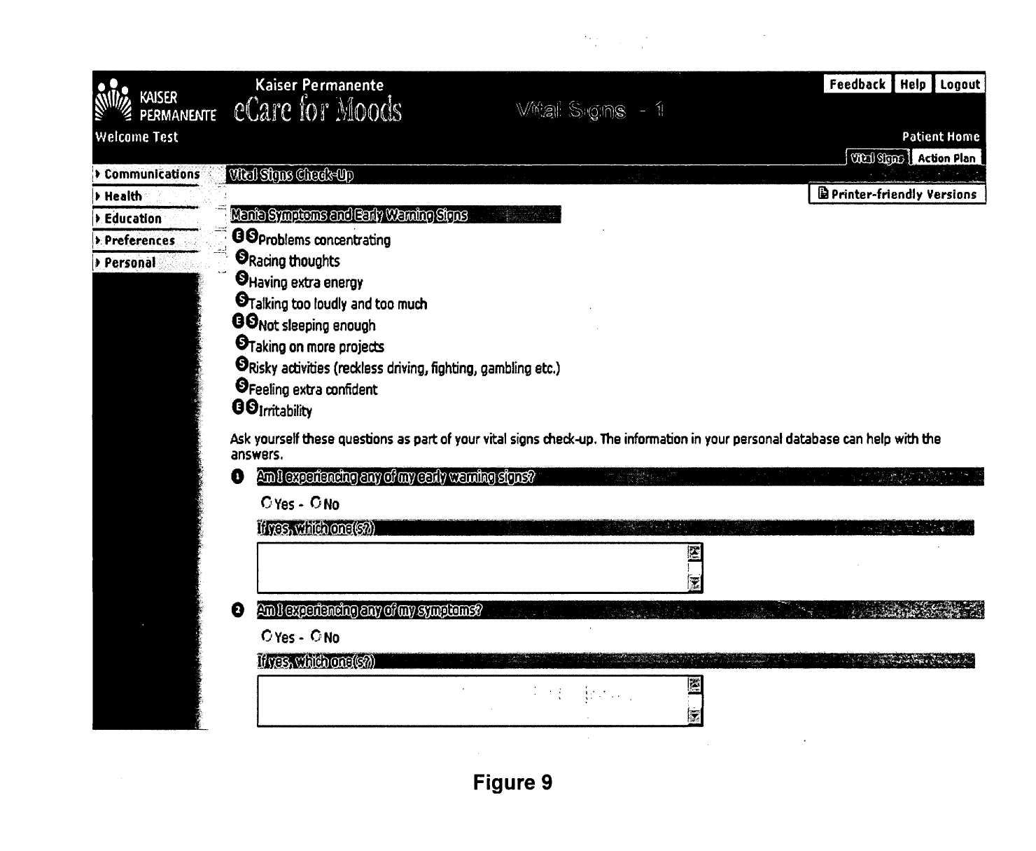

FIG. 9 illustrates a webpage for monitoring patient's vital signs according to an embodiment of the present invention. The patient is shown how to monitor his own vital signs in the education course. The webpage provides a list of symptoms and early warning signs to help the patient to monitor the vital signs. A patient may also monitor the vital signs by filling out questionnaires on a regular basis. Clicking on the Vital Signs button brings up a screen that the patient may print out. Education on how to use this form is provided during the education course and in consultation online with the nurse.

### Managing Action Plan

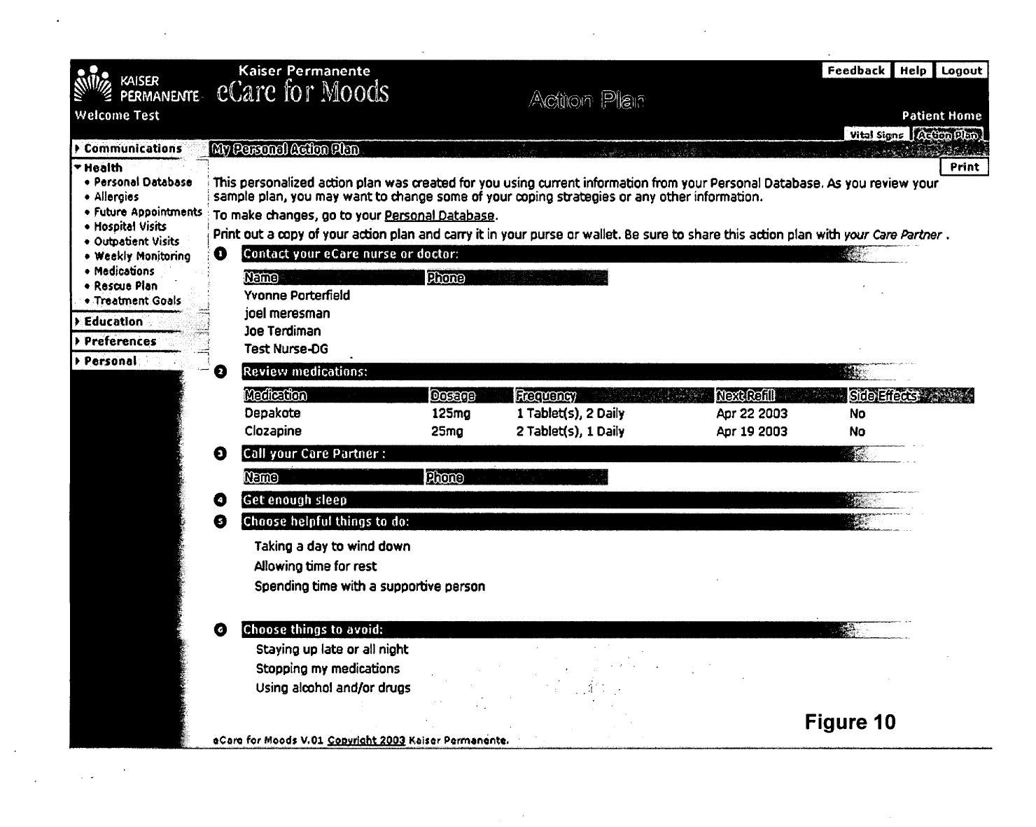

FIG. 10 is a webpage showing a patient's Action Plan according to an embodiment of the present invention. The patient may click on the Action Plan button to view the details of the Action Plan. The Action Plan includes contact information of healthcare providers and care partner. In addition, the Action Plan includes specific helpful action items, such as a list of helpful things to do and a list of things to avoid for the particular patient. It can also include instructions about medication use, or information on when to seek immediate care if there are triggers, early warning signs, or intensified symptoms. The Action Plan contains key information that the patient may printout and keep. A printer friendly version is available by selecting Print at the top right hand corner of the plan.

### Patient Assistance

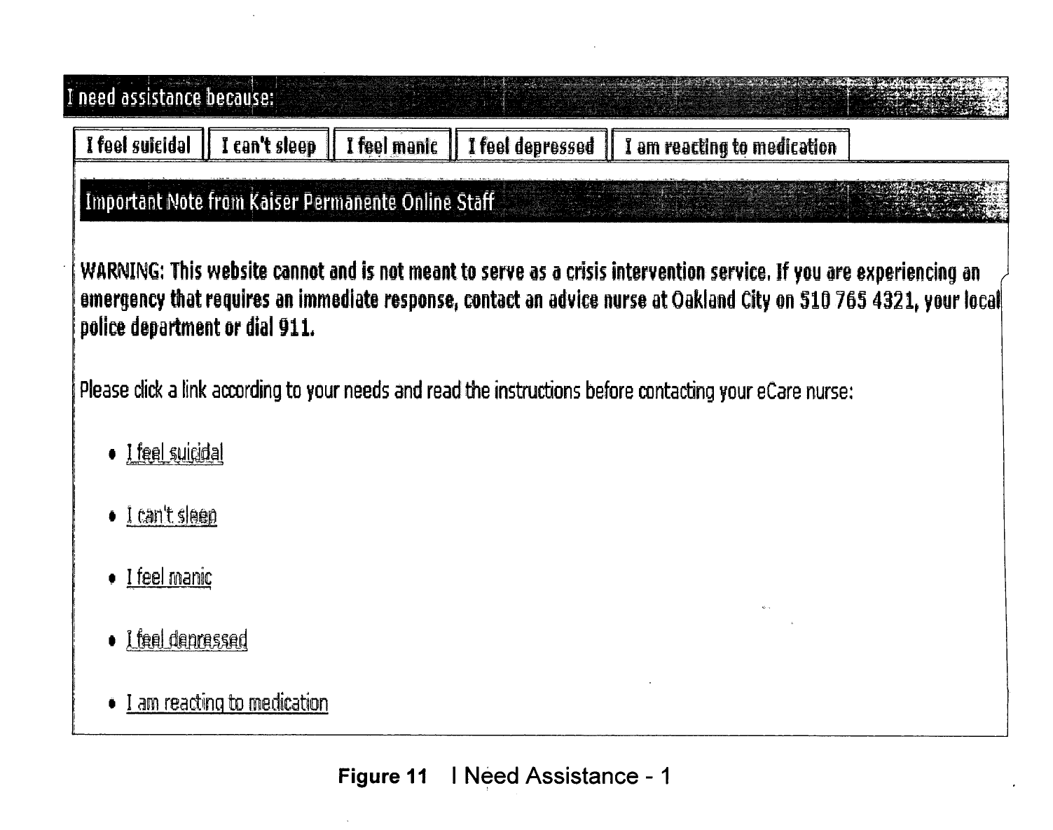

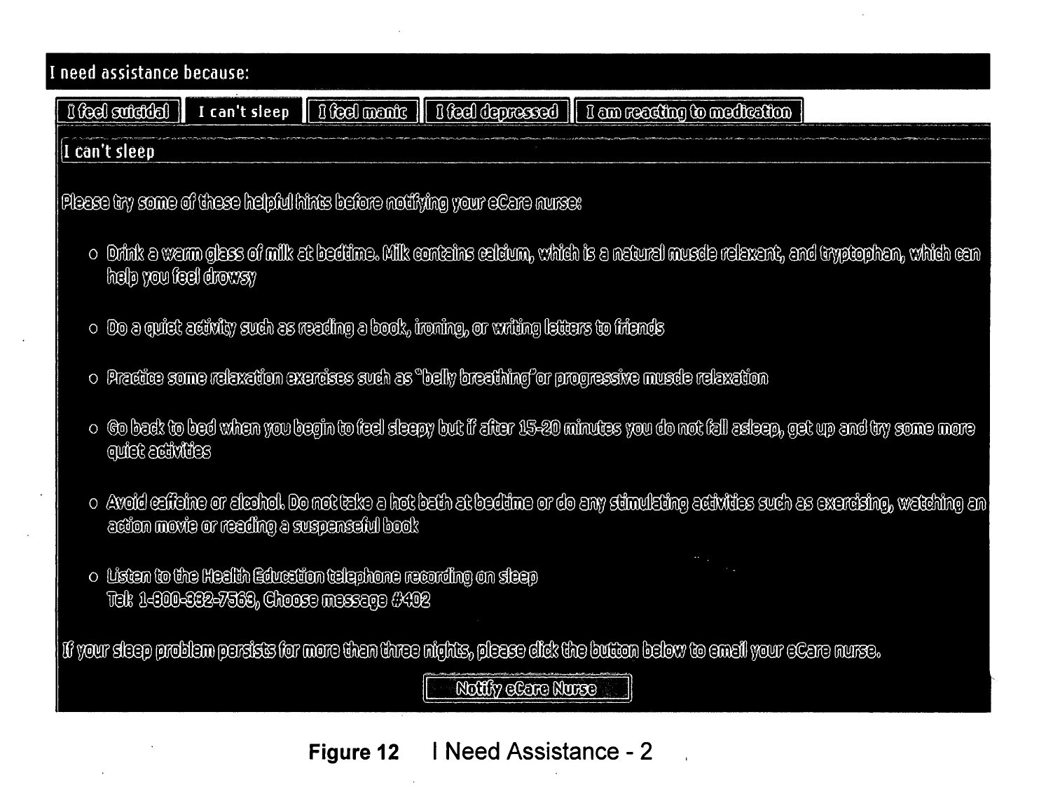

FIG. 11 illustrates a webpage for helping a patient in need of assistance according to an embodiment of the present invention. The I Need Assistance page allows a patient to access help on different topics. These topics can be accessed by the tabs at the top of the page or by the topic hyperlinks. Each assistance topic may provide the patient with relevant information, and advise the patient on activities that the patient may do to help himself. FIG. 12 illustrates a webpage for helping a patient within a topic of I Need Assistance according to an embodiment of the present invention. The webpage provides a number of suggestions to a patient who has the problem of I Can't Sleep. If the problem persists, the patient may contact a healthcare provider by clicking the Notify eCare Nurse button.

### Alert Generation

Alerts are based on configurable criteria defined for the monitored variables and/or the individual patient. Alerts are sent to the provider and appear on the provider's home page, which shows a list of action items for the provider's panel of patients. Alerts are typically generated when physiological measurements are outside a set of preset limits. For example, the patient's answers to the monitoring questions have certain specified values, and/or the patient has failed to refill a medication or to complete a scheduled task. For example, the patient fails to use the website within a specified period of time.

### Calendar and Task List

A calendar is built into the eCare system. Patients use the calendar to keep track of various scheduled tasks that are part of the treatment protocol (e.g., exercise for 1 hour on Monday, Wednesday, and Friday). A task list is provided to keep track of unscheduled tasks, such as refilling a prescription.

### Treatment Protocol

A graphical display of the treatment protocol and the patient's treatment status relative to the protocol is created. This online display helps providers to plan treatment of the patient according to the treatment protocol and to follow the course of treatment over time.

### Security

The eCare system includes a web server, which is located outside the institution's firewall. The web server is accessible from the Internet and it uses a Secure Socket Layer (SSL) 128-bit encryption to maintain security of data transmission. A Public Key Infrastructure (PKI) certificate may be obtained from a recognized certificate authority (e.g., Verisign, Inc). Only patients, care partners, and providers with accounts on the system may be able to access the web site. Patient data are stored in a relational database server inside the firewall. The data may be transmitted to the external site when the patient's record is accessed. The data is temporarily cached on the web server, and it is deleted when the user's session terminates. An internet protocol (IP) and a port specific tunnel through the firewall connect the web server to the database server and allow secure communication between the two. FIG. 1 illustrates various security measures taken by the eCare system, including an internal firewall and an external firewall.

### Communication Methods

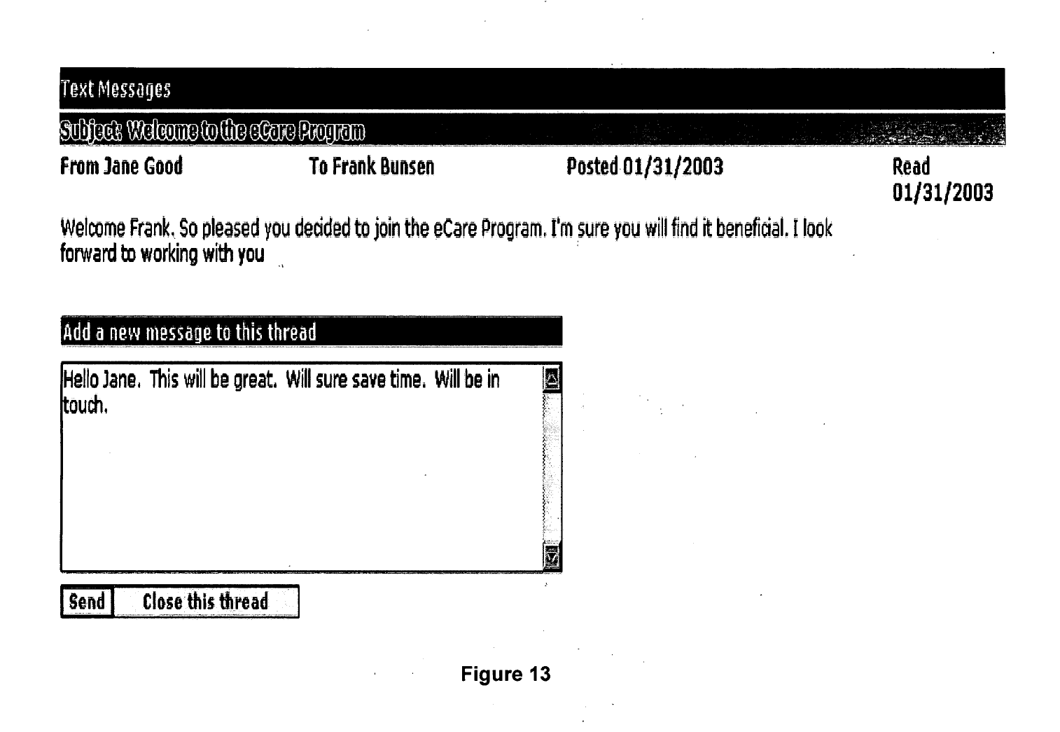

The eCare system implements the following communication methods:
a. Secure messaging: secure, encrypted messages may be sent between patient and provider in either direction at any time. All messages are saved in the database. An additional, unencrypted email message may be sent to the user's public email address with notification that a message has been delivered to the secure web site. FIG. 13 illustrates a webpage for sending secure messages between a patient and a healthcare provider according to an embodiment of the present invention. Any new message a patient has received is displayed on the homepage. A patient may view and reply to the message by clicking on a View button (not shown). Once a reply message is composed, the patient may click on a Send button to send the reply message. If the patient feels that the topic of his message/messages has been covered, he may end the discussion by clicking on the Close This Thread button. The messages may still be viewable but may not be opened for further discussion.
b. Instant messaging: one method of enabling real-time communication between patient and provider is through secure instant messaging. To limit the number of simultaneous conversations, the system requires the provider to initiate the communication. A person skilled in the art would recognize that either the provider or the patient may initiate the communication.
c. Voice over IP: another method of real-time communication is Voice-over-IP (VOIP). This method is useful for patients with a single telephone line who are connected to the Internet through that telephone line with a modem, and want to talk to their provider while they are connected to the eCare web site.
d. Video-over-IP: the eCare system may apply Video-over-IP in delivering health care services.
e. Discussion forums: the eCare system supports moderated and unmoderated discussion forums. Participation in these forums, especially the forums moderated by providers, is a useful educational tool for the patients.

### Legacy Interfaces

Many institutions have some form of electronic medical records (EMRs) that contain clinical information that may be relevant to the management of patients' healthcare, such as medications prescribed, significant health problems, allergies, etc. In order to seamlessly display EMRs together with information captured by the eCare system, an application interface is developed to facilitate downloading EMRs into the telemedicine system. Examples of protocols supported by the eCare system include Extensible Markup Language (XML) and direct SQL calls.

### Care Partner

A key component of the eCare system is the active support of a care partner, designated by the patient. A care partner is a person designated to participate in the care of the patient, including parents, guardians, a family member, or a friend. The care partner is given an account on the eCare system, and has the ability, with the patient's consent, to view certain portions of the patient's record and participate in the same educational programs. When patients have the support of care partners with whom health related issues can be discussed, patients may be more likely to more effectively self-manage their disease, which may lead to better care of the patient and better clinical and functional outcomes.

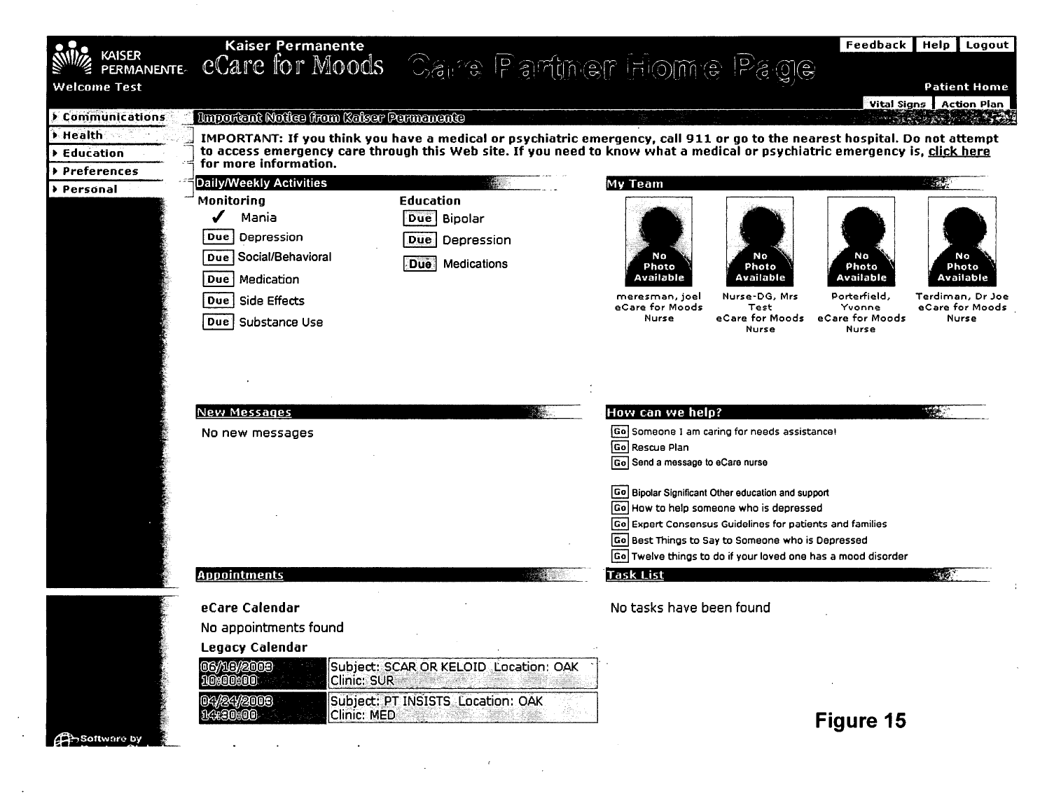

FIG. 15 illustrates an implementation of a care partner home page according to an embodiment of the present invention. The care partner home page provides information about monitoring activities, medical personnel contacts, messages, upcoming appointments, task lists, and educational materials. The care partner may monitor signs and symptoms of the patient and provide inputs to the eCare system. The signs and symptoms include medication use, medication side effect, substance use, as well as mania, depression, social depression, and social behavior of the patient. The care partner may receive messages from the patient, view past and current appointments of the patient, and view his own task list. These activities of the care partner are performed with the patient's permission.

Through the web pages provided by the eCare system, the care partner may provide inputs regarding the patient by sending messages to medical personnel reporting situations when the patient needs help. In addition, the care partner may communicate with the medical personnel about triggers, use of coping strategies, disease prevention strategies, and any other patient characteristic with respect to the treatment of the chronic disease. In return, the medical personnel may send feedback to the care partner's inputs and reward the care partner for tasks successfully performed. Alerts may be sent to the care partner when certain signs and symptoms he entered into the eCare system should be concerned about or noticed, such as a positive and unanticipated response to a treatment or medication or coping strategy.

The educational materials include tailored education for the care partner. The educational program includes online, interactive educational sessions for different conditions of the patient that a care partner may encounter. In particular, the care partner can take interactive courses in bipolar disorder, depression, and their related medications by clicking the course topic shown on the left hand side of the web page. The educational materials also include linkages to selected websites on topics of particular interest, such as expert consensus guidelines for patients and families, best things to say to someone who is depressed, and twelve things to do if your loved one has a mood disorder. The information is available 24 hours a day and it is all neatly organized in one place and on one web site so the care partner does not have to search all over the Internet for such information. A point system may be established where the care partner can be rewarded for completion of certain sections of the program.

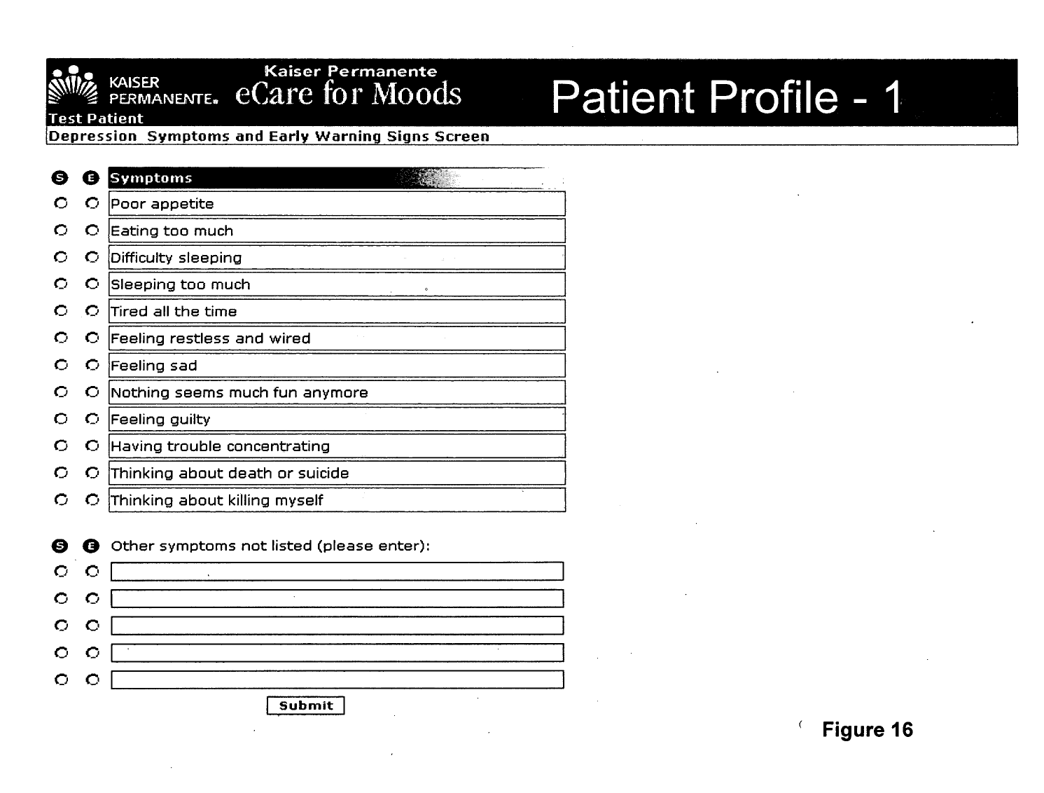

FIG. 16 illustrates a profile of the patient that may be provided by a care partner showing depression symptoms and early warning signs according to an embodiment of the present invention. Considering the case of monitoring a child taking an SSRI for depression, the parent, who knows the child best, can set up a profile in the eCare system that allows recording of behavior changes in the child, but put in the context of what is normal behavior for this particular child. Then, any change in behavior of the child with respect to relating to other people can generate an alert to the parent and to a clinician at the same time. For example, the child begins spending most of the time alone in their room. For some children this may be just a condition of a normal style of interaction. They may be loners, or working on certain school projects, or playing games with their friends over the Internet. For children who are generally quite gregarious, this may represent a major change in behavior and a signal for concern. By providing a way for the care partner to enter the patient profile online, the eCare system can address the issues described in the above example by providing an individualized monitoring of the patient at home by someone who really knows the patient.

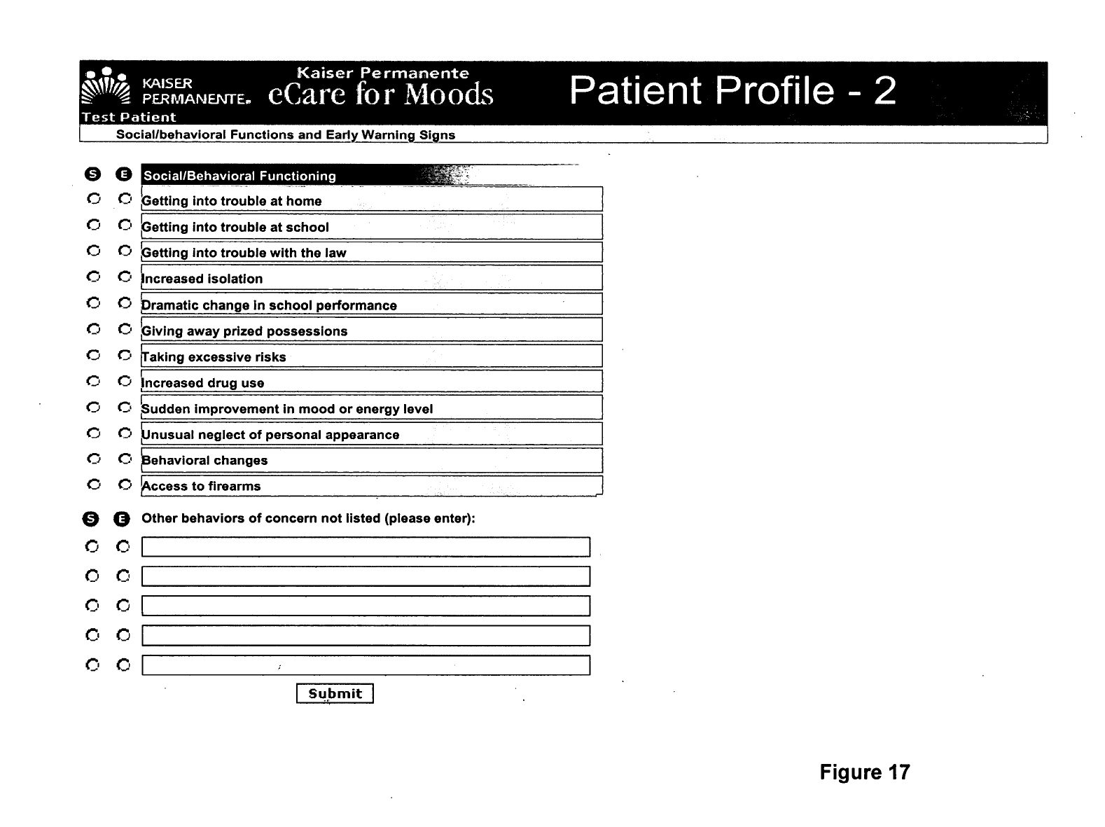

FIG. 17 illustrates a profile of the patient that may be provided by a care partner showing social and behavioral functions and warning signs according to an embodiment of the present invention. This profile page shows an individualized profile of behaviors that indicate changes that would be of concern. The behaviors listed may include those that are generally considered indicators of suicidal behavior in anyone and individual behaviors that would be of concern if observed in this particular patient. The frequency of monitoring can be as often or as infrequent as determined appropriate with no significant increase in cost to the eCare system.

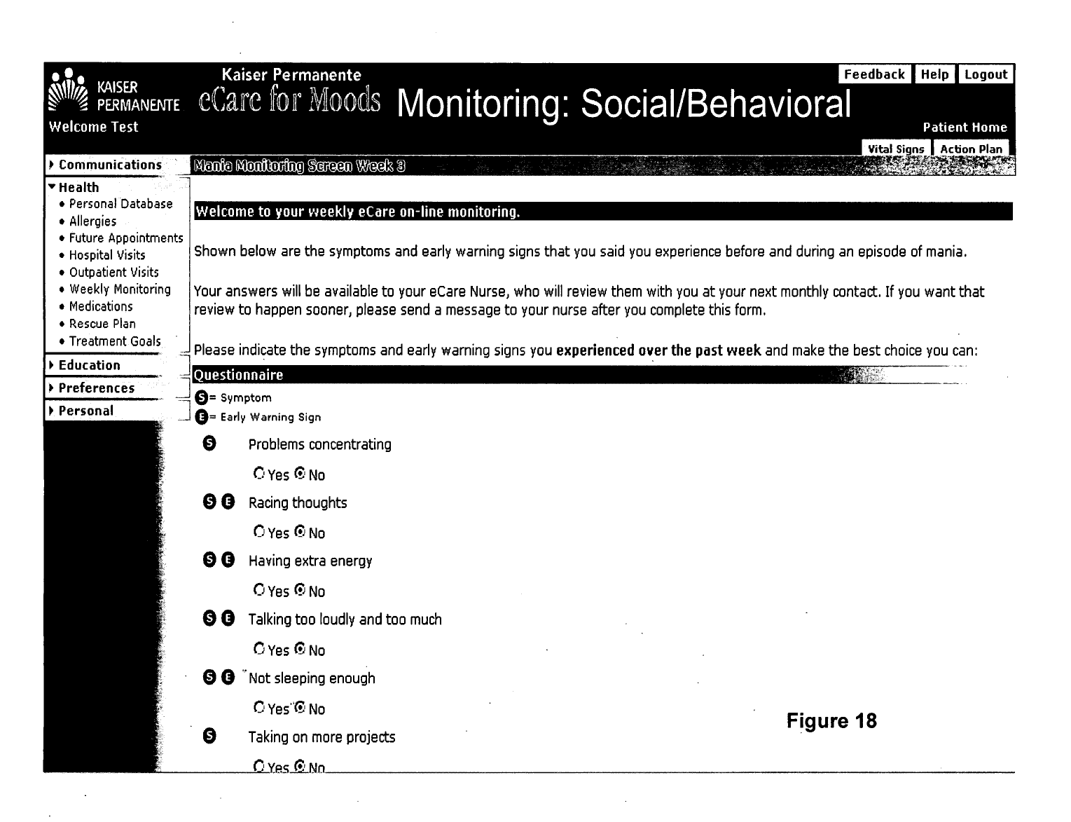

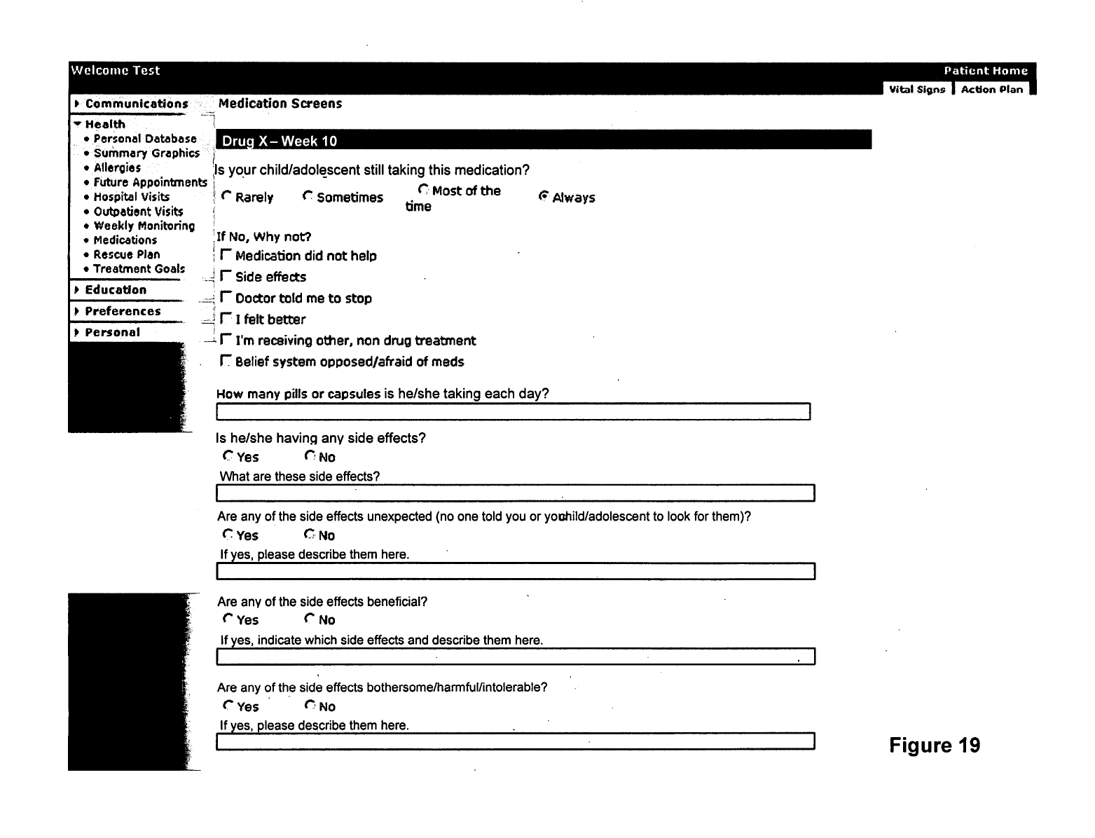

FIGS. 18 and 19 provide two specific examples for monitoring a patient in conjunction with the patient profiles shown in FIGS. 16 and 17. Specifically, FIG. 18 illustrates a web page for monitoring social and behavioral signs of the patient according to an embodiment of the present invention. FIG. 19 illustrates a web page for monitoring medications and their related side effects on the patient according to an embodiment of the present invention. This monitoring may be important in testing new medications and their side effects from clinical trials through post-marketing studies, when both harmful and beneficial are not yet known yet. By involving the care partner in the patient's treatment process, the relationship between the patient and the care partner can be monitored and steps can be taken to avoid “burnout” of the care partner. Also, organized discussion forums can be set up through the eCare system to allow a care partner to share experience and information with other care partners in similar situation.

### Database

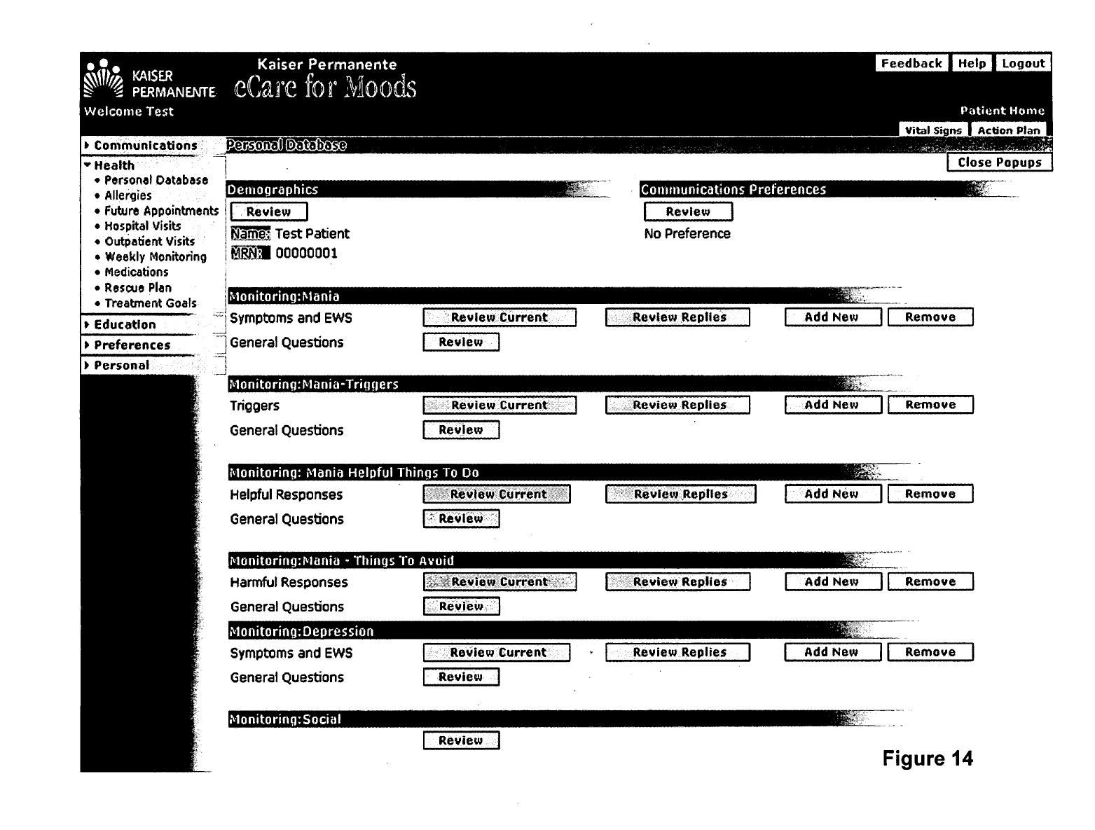

Clinical information in the eCare system is stored in an SQL-compliant database, behind the institution's firewall. The database may be configured to support clinical, administrative, and research purposes. To display clinical data on an individual patient, the patient's data is retrieved from the eCare database and/or from any legacy databases and is transferred to the web server for integration and display. FIG. 14 illustrates a webpage for managing a patient's personal database according to an embodiment of the present invention. The personal database webpage includes patient's demographics, communication preferences, and various monitoring functions. For example, the various monitoring functions may include monitoring triggers, symptoms, and early warning signs of a chronic disease such as mania. The database may also include a list of helpful things to do and a list of things to avoid.

### Administrative and Statistical Reporting

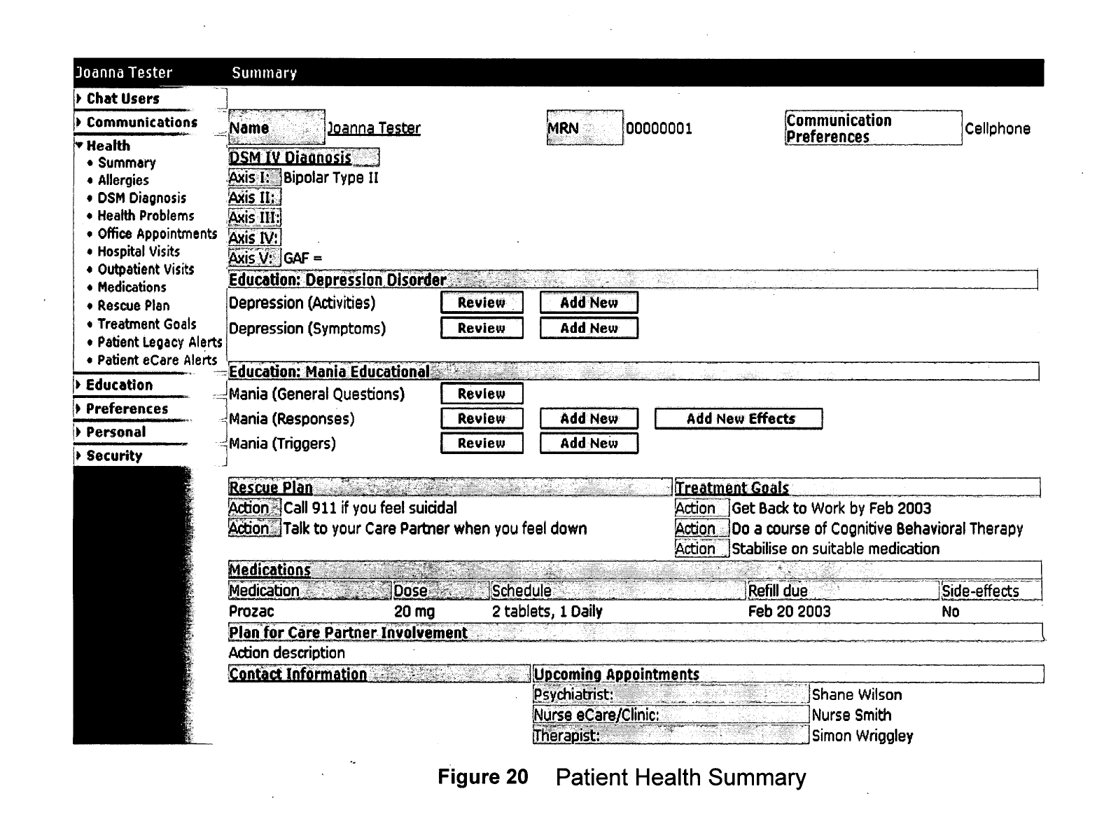

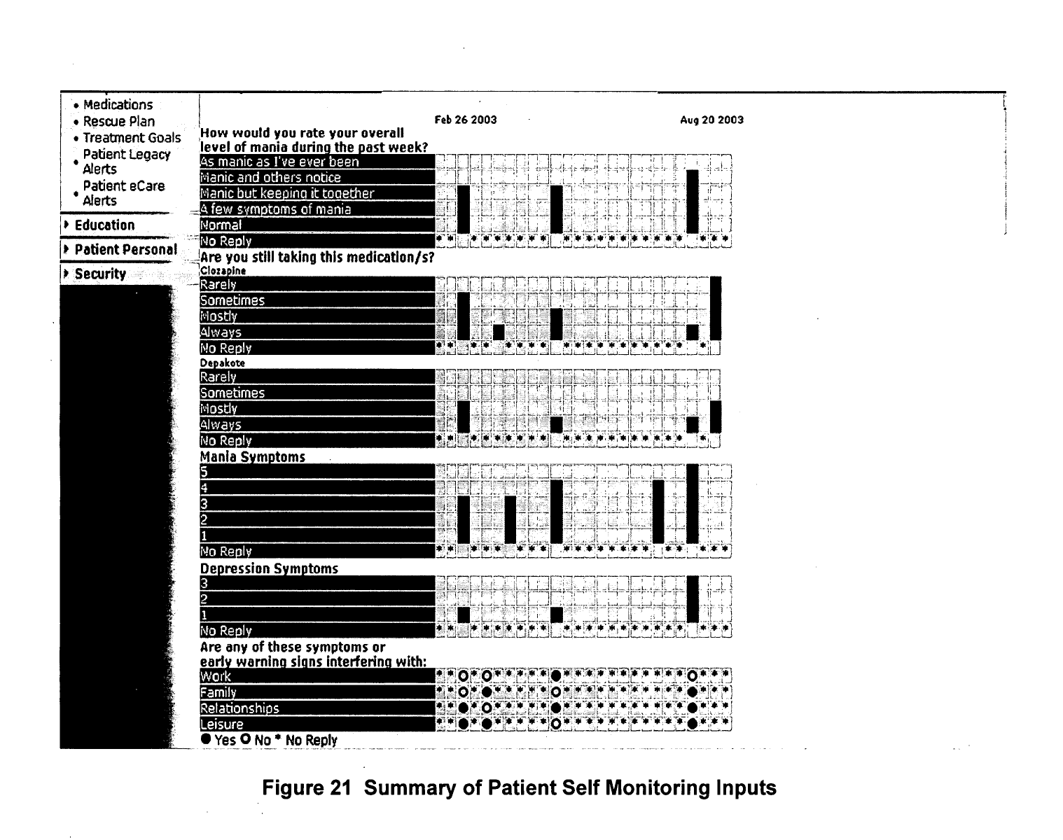

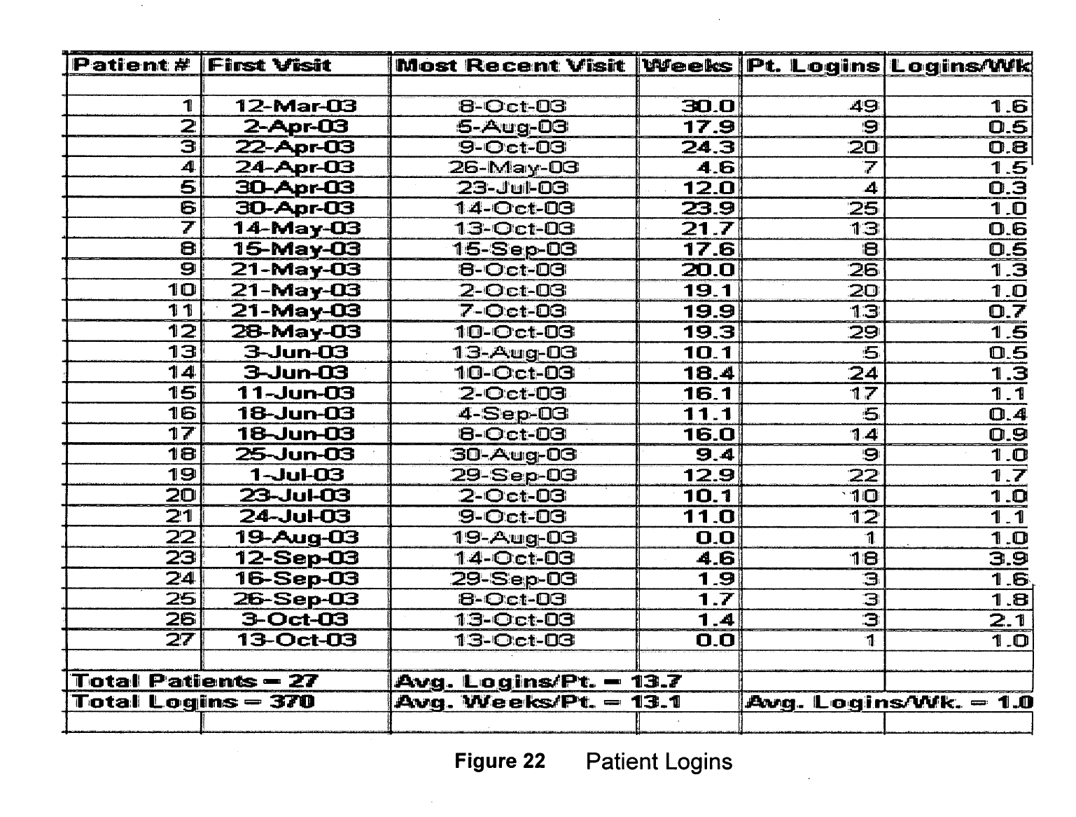

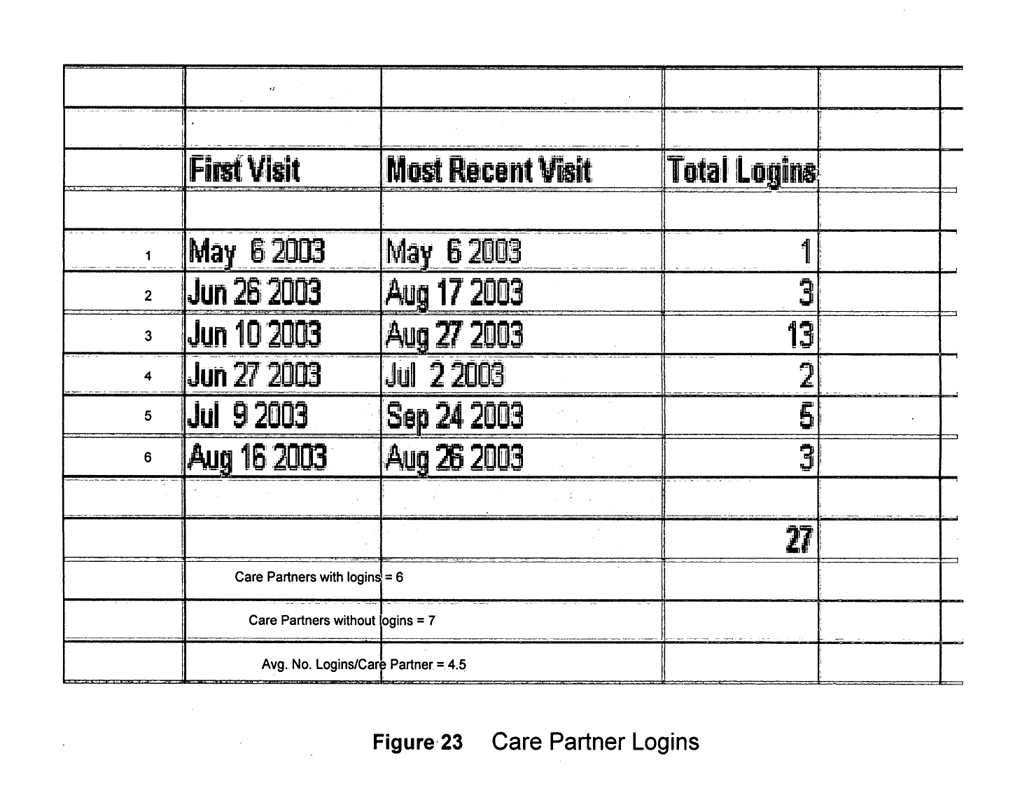

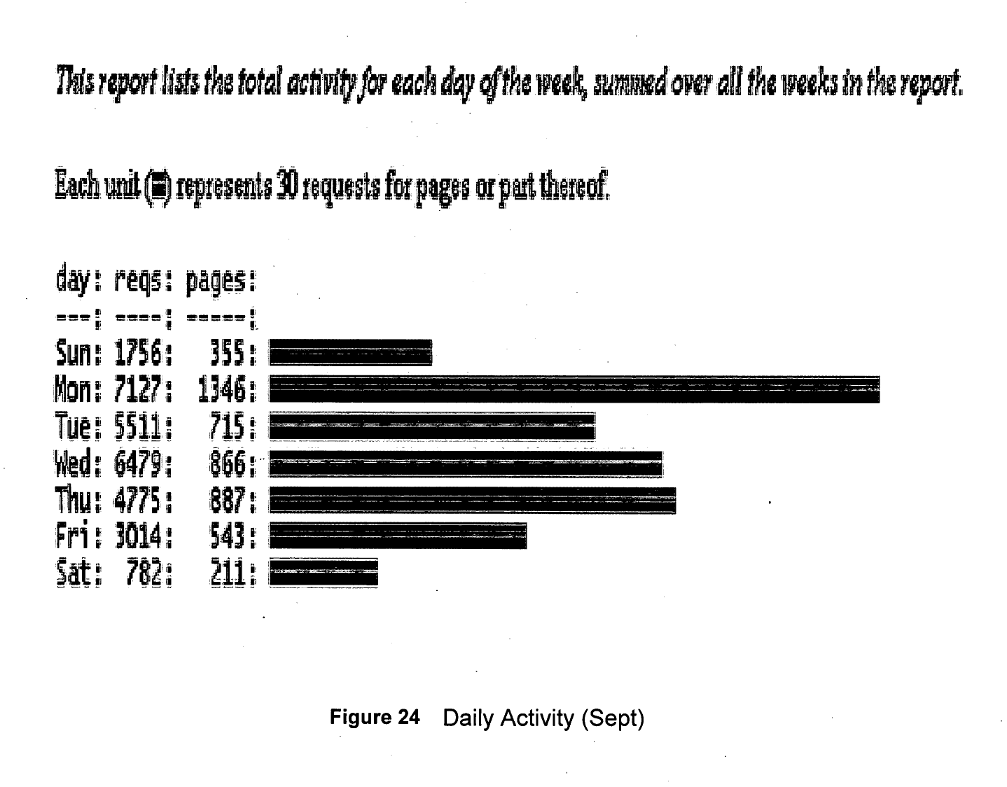

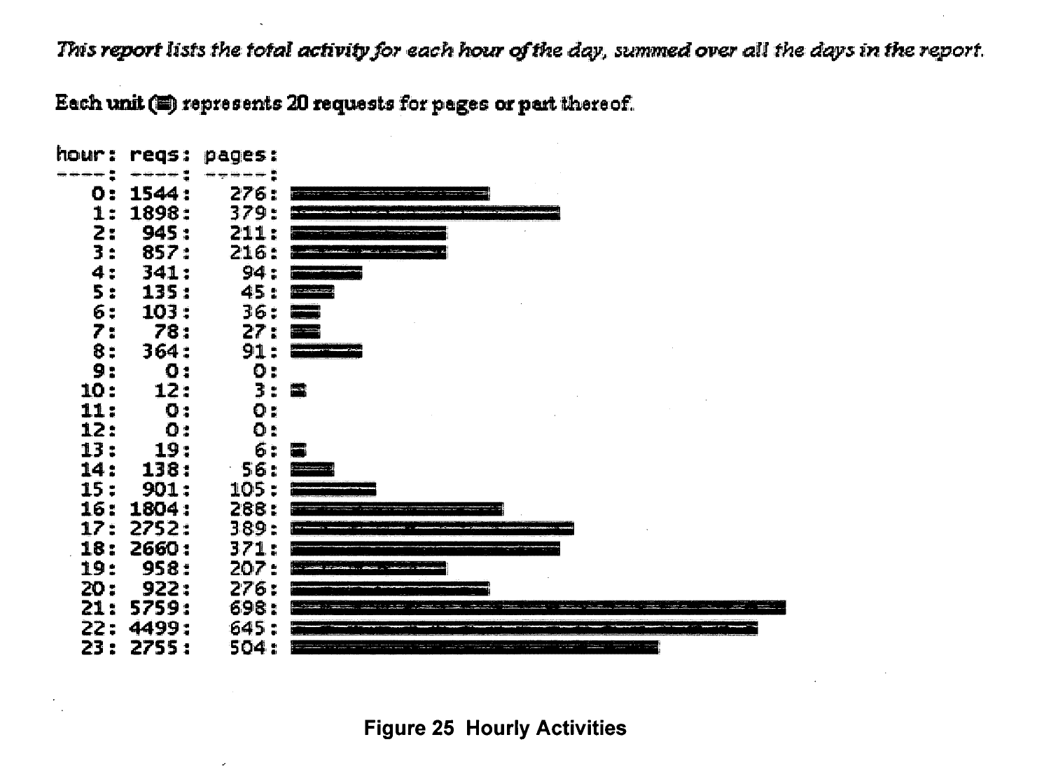

A number of built-in administrative and statistical reports may be developed. In addition, the database may be accessible to standard report generators and to ad hoc queries for research. Specifically, FIG. 20 illustrates a webpage for displaying patient's health summary according to an embodiment of the present invention; FIG. 21 illustrates a webpage for displaying a summary of a patient's self-monitoring inputs over a period of time according to an embodiment of the present invention; FIG. 22 illustrates a webpage for monitoring patients' usage of the eCare system according to an embodiment of the present invention; FIG. 23 illustrates a webpage for monitoring a care partner's usage of the eCare system according to an embodiment of the present invention; FIG. 24 illustrates a webpage for monitoring patients' usage of the eCare system on a particular day of the week according to an embodiment of the present invention; and FIG. 25 illustrates a webpage for monitoring patients' usage of the eCare system on a particular hour of the day according to an embodiment of the present invention.

### Treating Obese Patients

In another embodiment, the eCare system may be configured to support a specific area of health care management, such as treating obesity. Obesity is an area of increasing prevalence in the U.S., and there is a need for an evidence-based intervention system to prevent or reduce weight-gain in patients on anti-psychotic medications. The eCare system can be configured for this intervention system, which employs periodic monitoring of weight, blood glucose, treatment adherence, education, and behavioral modification through psychosocial interventions. The patient and provider satisfaction may be measured at a predefined intervention. Changes in patients' weight, blood sugar levels, and functional status from baseline may also be measured and monitored. The following describes a number of intervention methods for treating obesity according to an embodiment of the present invention.

In one implementation, the eCare system includes an online, interactive education course with weekly sessions. Each session includes a worksheet that may help patients to understand their own experience with obesity. Other sessions are designed to help them develop individualized action plans that put several weight management strategies together and help them identify alternative ways of coping with stressful situations. In particular, the interactive education course includes the following topics.
a. Health information: health information about the risks of obesity, the role of genetics, how to self-monitor (weight and blood glucose), when to get help, and an overview of the course.
b. Reduction of intake of fats and sugar, and increased consumption of fruits and vegetables: patients, care partners, and other family members are given information on nutrition, emphasizing ways of implementing a well-balanced, low-fat, low-sugar diet.
c. Patterned eating: in order to combat increased hunger, the importance of a regular, predictable eating pattern consisting of three meals and two snacks daily may be emphasized.
d. Stimulus control designed to reduce non-planned eating episodes: major interventions include planned shopping to keep problem foods out of the home, proper food storage, and limiting the cues associated with eating (e.g., not eating while watching TV).
e. Daily exercise: non-programmed ways of increasing caloric expenditure are encouraged including use of stairs, parking further than necessary from a destination, and doing laborious household chores, such as mowing the lawn.
f. Learning to cope with urges to eat: patients and families are instructed in how to cope with urges to eat associated with appetite changes. These include delay, distraction, and use of very low-calorie snacks when other measures fail.
g. Using the web site to record individualized action plans: individualized action plans can be accessed in times of stress or under other situations that may lead to weight gain (e.g., change in a medication).

Patients are asked to measure their weight and blood glucose weekly at home, using scales and glucometers provided by the study. Through interfaces to the instruments, the measurements are uploaded automatically to the web site. Calibration of each scale and glucometer is performed prior to delivery to the patient.

Patients are encouraged to set weekly goals that they can accomplish with respect to healthy eating, increased exercise, and coping strategies, so that they can experience success. A provider, such as a health educator or a nurse, may offer support, encouragement, and praise when they accomplish these goals and help them set additional ones. The provider may also help them overcome barriers and track their general health, appetite, diet, exercise, and medication side effects. When patients gain weight, the provider may take the treatment regimen to the next step in a stepped-care program described below. Patients are encouraged to record their successful strategies in a personal database that is part of the web site.

An individualized plan is developed with each patient based upon their health and current exercise patterns. In addition, a care partner may exercise with the patient, or may provide praise and advice where appropriate.

A care partner (e.g., a family member) is given an account on the system and encouraged to support the patient's efforts to lose weight.

Alerts may be generated if certain predetermined monitoring parameters for an individual patient are exceeded. For example, an alert may be generated if there is a weight gain of 2 kilograms (kg) above baseline weight or if fasting blood sugar exceeds 126 mg/dl. Other alerts may be specified for individual patients, depending on their health and functional status.

The weight control strategies outlined above may be implemented as part of a stepped-care program, which varies in intensity according to actual weight gain. Step One care includes one initial clinic visit and regular visits to the eCare web site. Step Two care includes more frequent visits to the web site and real-time communication with a clinic nurse. Step Three care includes weekly communication with the clinic nurse, in-person visits to the clinic, and a more structured eating and exercise plan.

Care partners are encouraged to support patients in their efforts. Nurses may monitor patients' progress via the eCare web site. As a supplement to the online education program, patients are also encouraged to attend classes that address weight control issues and take advantage of weight control services offered in the community.

The criteria for step changes are linked primarily to weight change. Step One care is provided to all intervention patients, although those with a diagnosis of diabetes also receive Step Two care immediately. Other patients receive the additional components of Step Two care if they gain two kg (4.4 pounds) above their baseline weight, or if they ever show a fasting blood glucose level of 126 mg/dl or above, the level indicating diabetes according to the

American Diabetes Association. If a patient shows a gain of 5 kg (11 pounds) over baseline, they may receive care at the Step Three level.

For patients at level one, contents may emphasize reduction of fats and sugar, patterned eating, stimulus control, exercise, behavior activation, and brief supportive counseling. Weight management strategies may be framed in terms of “maintaining good eating and exercise habits” rather than as a problem. For patients at level two, there is increased emphasis on weight management strategies, and continued attention to reduction of fats and sugar, patterned eating, stimulus control, behavior activation, and brief supportive counseling. For patients at level three, the focus is on weight management strategies and quick medical attention.

Providers need to be aware of any other services that patients are receiving, significant health problems, and all medications, through the legacy interface to the obesity management system. They may tailor their weight management strategies to take these other items into account.

There are number of benefits achieved by the disclosed eCare system. First, it provides new paradigms of health care delivery that both providers and patients may prefer to use. These paradigms include such features as home monitoring, patient education, customized care, electronic communication, etc., that can be integrated into a seamless web-based package. For chronic conditions in which routine monitoring can be done at the patient's home, the eCare system may reduce the need for clinic visits, day care, parking, etc. In addition, the eCare system provides an effective, online, interactive education program, customized to the individual patient, with links to relevant web sites that can be accessed at the patient's leisure. Moreover, the eCare system provides insights and information that may improve a patient's compliance with treatment protocols. Furthermore, the eCare system can generate alert messages that are sent automatically to providers, when a patient's health status requires intervention. Instead of the usual telephone tag and delays patients and providers often experience when they need to communicate, a secure messaging system or instant messaging can often save time at both ends.

One skilled in the art will recognize that the eCare system may be modified to function as a more general system that can be configured by a health care institution for delivering health information (e.g., patient education on a variety of topics) and managing other chronic conditions and diseases (e.g., diabetes, hypertension, asthma). The eCare system is designed to minimize modifications required to configure the software for managing a new disease area. It is primarily table driven, and supports importing content of web pages, menus, tables, business logic, and database schemes. The eCare system includes functionalities such as home physiological monitoring (e.g., weight, blood sugar, blood pressure, etc.), display of treatment protocols, and patient's treatment status relative to a protocol. Furthermore, the eCare system is designed to increase the portability of the application to a wider variety of home computer platforms and operating systems as well as wireless and hand-held devices. Examples of common protocols supported by the eCare system are XML and direct SQL queries.

One skilled in the relevant art will further recognize that many possible modifications and combinations of the disclosed embodiments may be used, while still employing the same basic underlying mechanisms and methodologies. The foregoing description, for purpose of explanation, has been written with references to specific embodiments. However, the illustrative discussions above are not intended to be exhaustive or to limit the invention to the precise forms disclosed. Many modifications and variations are possible in view of the above teachings. The embodiments were chosen and described to explain the principles of the invention and their practical applications, and to enable others skilled in the art to best utilize the invention and various embodiments with various modifications as are suited to the particular use contemplated.

1-18. (canceled)

19. A system for providing patient-specific healthcare to a patient having a chronic disease, medical condition, or health risk, the system comprising:
a computer server configured for creating one or more web pages comprising (i) a predefined list of symptoms or early warning signs relating to the patient's chronic disease, medical condition, or health risk, (ii) input fields configured to receive from the patient additional symptoms or early warning signs relating to the patient's chronic disease, medical condition, or health risk, and (iii) input fields configured to receive from the patient as to whether the patient is experiencing a listed symptom or early warning sign;
a patient computer configured for (i) receiving, from the computer server via the internet or a network, the one or more web pages; (ii) displaying the one or more web pages on a graphical display; (iii) receiving, from the patient, input as to whether the patient is experiencing the predefined symptoms or early warning signs; (iv) receiving, from the patient, additional symptoms or early warning signs relating to the patient's chronic disease, medical condition, or health risk, (v) receiving, from the patient, additional input as to whether the patient is experiencing one of the additional symptoms or early warning signs; and (vi) transmitting, via the internet or the network, the input and additional input to the server computer; and
a database server configured for storing patient-specific and patient-identifiable information comprising the input and additional input,
wherein the server computer automatically sends to the patient computer a patient-specific treatment recommendation for the patient based at least in part on the input and additional input.

20. The system of claim 19, wherein the database server includes a legacy server for storing the patient's medical or medication records.

21. The system of claim 19, wherein the web page further comprises a home page for the patient for linking one or more items selected from the group consisting of signs and symptoms, daily activities, weekly activities, individualized action plan, healthcare team members, messages, appointments, medications and task list.

22. The system of claim 1, wherein the computer server further provides an interactive patient-specific education program according to the input and additional input provided by the patient, wherein the interactive patient-specific education program comprises customized web contents prepared in response to the input and additional input provided by the patient.

23. The system of claim 19, wherein the computer server further provides an interactive patient-specific self-management tool according to the input and additional input provided by the patient, wherein the interactive patient-specific self-management tool is adaptive to changes in the patient's knowledge of the chronic disease, medical condition, or health risk and to changes in the signs and symptoms.

24. The system of claim 19, wherein the patient computer is further configured for:
implementing a patient specific treatment plan;
providing an action plan or task list for informing the patient of things to do in response to the input and additional input provided by the patient; and
providing a calendar for keeping track of scheduled treatment activities.

25. The system of claim 19, wherein the patient computer is further configured for:
prioritizing healthcare resources according to the input and additional input provided by the patient; and
organizing the workload of healthcare providers using the input and additional input provided by the patient.

26. The system of claim 19, wherein the computer server further comprises generating automatic alerts to medical providers if the input or additional input provided by the patient deviates from at least one of a set of predefined parameters.

27. The system of claim 26, wherein the set of predefined parameters comprises at least one of the following conditions:
patient fails to monitor the signs and symptoms of the chronic disease, medical condition or health risk on schedule;
patient reports side effects from medication;
patient fails to take a medication;
patient experiences a worsening of symptoms;
patient experiences a new symptom;
patient fails to take medication as prescribed;
patient reports a change in side effects from medication;
patient fails to refill a prescription; and
patient fails to use the website within a specified period of time.

28. The system of claim 19, wherein at least some of the treatments are further customizable by a medical provider and stored in the personal profile.
